(19) United States
(12) Patent Application Publication
(10) Pub. No.: US 2006/0024744 A1
(43) Pub. Date: Feb. 2, 2006

(54) Methods for substantially simultaneous evaluation of a sample containing a cellular target and a soluble analyte

(76) Inventors: Rhonda A Mills, Pembroke Pines, FL (US); Jorge A. Quintana, Tavernier, FL (US); John A. Maples, Miami, FL (US); Paul M. Scibelli, Miami, FL (US); Wataru Hashimoto, Kanagawa (JP)

Correspondence Address: BECKMAN COULTER, INC.;C/O DLA PIPER RUDNICK GRAY CARY US LLP, 4365 EXECUTIVE DR, SUITE 1100, SAN DIEGO, CA, 92121-2133, US

(21) Appl. No.: 10/515,073

(22) PCT Filed: Jul. 28, 2004
(86) PCT No.: PCT/US04/24235

§ 371 Date: May 10, 2005

(51) Int. Cl. G01N 33/53; G01N 33/567

(52) U.S. Cl. 435/7.1; 435/7.2

(57) ABSTRACT

Methods are provided for monitoring treatment of a subject using heparin therapy or a ligand, such as an CD20 or CD52 antibody, that binds to a cellular marker and to which the subject may develop masking reactions or auto-antibodies that inhibit evaluation of treatment. The methods involve adding to a single container a sample containing cells that express the cellular target with (i) a soluble ligand that binds the cellular target, (ii) a soluble ligand that binds the soluble analyte or a competing soluble analyte; and (iii) a capture medium that binds directly to the soluble analyte, indirectly to the soluble analyte, or to the soluble ligand that binds to the soluble analyte. Complexes formed in the container by interaction of these components are substantially simultaneously analyzed and quantitated without physically separating the complexes. Kits for performing the assays are also provided.

### BACKGROUND OF THE INVENTION

The present invention relates generally to assay methods for quantitative and qualitative evaluation of biological samples and more particularly to assay methods for biological samples containing both cellular and soluble targets or analytes.

The ability to detect and/or measure a wide variety of targets, analytes, molecules, chemical compounds and complexes and the like in a variety of biological samples or products has significant use the diagnosis of disease, the treatment of disease, the monitoring of the efficacy of therapy, research in molecular biology, the detection and monitoring of water purity, product contamination, and other fields. A number of different types of assays have evolved depending upon the identity and state of the target to be analyzed, e.g., a target immobilized on or in a substrate (such as a cell) or a soluble target, a target that is proteinaceous, or a chemical compound, etc. Thus, immunoassays exist that identify proteinaceous targets using antibodies; competition immunoassays identify targets by allowing the target to compete for binding to a limited amount of an antibody with a known amount of a labeled antigen. The amount of labeled antigen bound to the antibody is inversely proportional to the amount of antigen in the sample. Immunometric assays employ a labeled antibody and the amount of labeled antibody associated with the target is measured as directly proportional to the amount of target available in the sample. Cytometric assays identify targets by size, shape, charge, light diffraction or reflection, or other means.

Generally, assays to detect targets by immobilizing a ligand on a solid support and forming a complex between the target and the bound ligand require different processing steps than assays employing soluble ligands to detect. Further, in assays performed on blood samples, phagocytosis of the solid support particles by myeloid cells in the sample can introduce error into the assays. Most known assays also involve multiple processing steps, e.g., lysing, washing, and physically separating components formed in the sample prior to detecting the appropriate ligand or label. Further known assays generally employ a wash step to remove bound analyte from soluble analyte and are performed in a serum, plasma or media matrix devoid of cells.

Additional steps of washing, lysing and separating introduce inaccuracies by reducing the amount of target inadvertently. The need to perform a number of different assays on a single rare or small sample is often another problem experienced with assays, which leads to depletion of the sample material and an increase in the costs of the assays themselves.

Various assays are described in the following references: Lindmo T, et al, J. Immunol. Meth., 1990 126:183-189; Frengen J et al, J. Immunol. Meth., 1995 178:131-140; Frengen J et al, J. Immunol. Meth., 1995 178:141-151; U.S. Pat. Nos. 4,572,028; 4,376,110; 5,006,459; 5,168,044; 5,426,029; 5,525,461; 5,567,627; 5,756,011; 5,811,525; 5,981,180; 6,268,155; and U.S. patent publication 2002/0076833.

Recent approvals of biologic therapies for treatment of cancer have opened up a new area for therapeutic monitoring. For example, treatment of B-cell lymphoma with monoclonal antibodies directed against CD20 (Rituximab®, Bexxar™) was FDA approved a few years ago. Initial results have been promising, however continued monitoring to evaluate remission versus tumor escape is required. After treatment, B-cells may no longer express CD20 due to B-cell depletion, tumor escape, or blocking of the CD20 either by the anti-CD20 treatment antibody (Rituximab, or Bexxar) or by circulating soluble CD20 that binds the detector antibody (Giles, F. J., et al. 2003. Br J Haematol 123:850-857).

For example, levels of circulating Rituximab® occur up to 6 months post-treatment and can block detection of CD20+ cells. The presence of cytoplasmic CD20 will indicate that Rituximab® is effectively blocking B-cell CD20, not that the B-cells have become CD20 negative (tumor escape) (Clarke, L. E. et al. 2003. J Cutan Pathol 30:459-462; Kennedy, G. A. et al. 2002. Br J Haematol 119:412-416; and Davis, T. A. 1999. Clin Cancer Res 5:611-615). The ability to detect circulating Rituximab® not only confirms the evaluation but also provides a quantitative measurement of the drug.

Currently, levels of circulating Rituximab® and CD20+ cells are assayed by flow cytometric, immunohistochemical and immunoassay methods separately. The ability to substantially simultaneously monitor both surface and cytoplasmic expression of these types of markers as well as the presence of circulating drug (Rituximab) would offer an important step forward in assessing therapeutic effectiveness.

Another example of a therapeutic monitoring application is the treatment of B-cell chronic lymphocytic leukemia with anti-CD52 antibody (CAMPATH-1, alemtuzumab), which was FDA, approved a few years ago. CD52 is a glycophosphatidyl inositol (GPI) anchored protein that is highly expressed on normal lymphoid cells and monocytes as well as on a large proportion of lymphoid cell malignancies—but not on hematopoietic progenitor cells (Dumont, F. J. 2002. Expert Rev Anticancer Ther 2:23-35). In treatment with anti-CD52 antibody, continued monitoring to evaluate remission versus tumor escape is required. After treatment, B-cells may no longer express CD52 due to B-cell depletion, tumor escape, blocking of the CD52 by the drug (CAMPATH-1) that binds the detector antibody ((Giles, F. J., et al. 2003. Br J Haematol 123:850-857). In addition the CD52 may be shed into the circulation, a characteristic of GPI anchored proteins.

The use of a second antibody with specificity for a different epitope of CD52 (e.g. HI186 or CF1D12) can assess tumor escape. The measurement of CAMPATH-1 serum levels can be used to optimize dose regimens, and also confirms the evaluation of tumor escape (Birhiray, R. E. et al. 2002. Leukemia 16:861-864 and Rebello, P. and G. Hale. 2002. J Immunol Methods 260:285-302). The potential for anti-idiotype antibodies, though less problematic when a humanized monoclonal antibody, such as CAMPATH-1, is used, may also be monitored [10]. In addition, due to the toxicity of this treatment resulting in extensive depletion of lymphocytes, the ability to quantitate differences in the level of CD52 expression may allow stratification of responders to non-responders [11]. To monitor this situation, analysis has historically been done separately using two to three different technologies, such as flow cytometry for surface expression and fluorescence microscopy and immunoassay for circulating CD52 or immune-complexes.

Auto-antibodies directed against the heparin/platelet factor 4 (H:PF4) complex are responsible for heparin-induced thrombocytopenia (HIT), an important side effect of heparin therapy (Reilly, R. F. 2003. Semin Dial 16:54-60). The incidence of HIT among patients receiving heparin is 1-3% (DeBois, W. J. et al. 2003. Perfusion 18:47-53). The increased thrombin generation is associated with decreased platelet counts (<150 k/μL) and high anti-heparin/PF4-antibody levels. This combination of events is potentially life threatening. The ability to rapidly determine the amount of anti-heparin/PF4-antibody could therefore help guide clinical management. Recently a new flow cytometric assay that detects anti-H:PF4 antibodies and platelet activation using Annexin V has been described (Gobbi, G. et al. 2004 Cytometry 58B:32-38. Previous to this development, beads coated with H:PF4 have been used alone to detect anti-platelet antibodies (Tazzari, P. L. et al. 2002. Transfus Med 12:193-198).

There remains a need in the art for more efficient methods of analyzing multiple analytes in a single sample, which methods can reduce the steps performed on the samples, thereby improving accuracy and cost effectiveness, while preserving rare samples.

### SUMMARY OF THE INVENTION

The invention provides high though-put methods for monitoring treatment of patients being administered a soluble ligand that binds to a cell marker. In one embodiment, the invention provides methods for monitoring treatment of a patient in need thereof with a treatment ligand that binds specifically to the cell surface expressed target CD20. The invention CD20 monitoring method includes, in a container containing a sample including bodily fluid having CD20⁺ cells obtained from the patient, performing one of the following:
i) incubating assay components in the container with a first soluble ligand that binds specifically to CD20 and is conjugated to a first distinguishable fluorescent label under conditions and for a time to allow formation of complexes of CD20 and the first ligand; or
ii) adding to the container a second soluble ligand that binds specifically to B cells and is conjugated to a second fluorescent label under conditions and for a time to allow formation of complexes between assay components; or
iii) the following combination of steps:
   a) adding to the container a capture particle linked to CD20 antigen;
   b) permeabilizing cells in the container; and
   c) incubating assay components in the container with a third soluble ligand that binds specifically to intracellular CD20 and is conjugated to a third distinguishable fluorescent label under conditions and for a time to allow formation of complexes of intracellular CD20 and the first ligand. Fluorescence from the fluorescent labels in complexes formed in the container is detected to monitor the treatment of the patient.

In another embodiment, the invention provides methods for monitoring treatment of a patient in need thereof with a treatment ligand that binds specifically to the cell surface expressed target CD20 by incubating together in a container under conditions and for a time sufficient to allow complex formation between the following assay components: 1) a sample comprising bodily fluid containing CD20⁺ cells obtained from the patient; 2) a first soluble ligand that binds specifically to soluble CD20 conjugated to a first distinguishable fluorescent label; 3) a second soluble ligand that binds specifically to B-cells conjugated to a second distinguishable fluorescent label; and 4) a capture particle linked to CD20 antigen. After permeabilizing cells in the container, the assay components in the container are incubated with a third ligand that binds specifically to intracellular CD20 and is conjugated to a third distinguishable fluorescent label under conditions and for a time to allow formation of complexes of intracellular CD20 and the third ligand, thereby forming a mixture of components therein. The presence of fluorescence from the first, second or third fluorescent labels in the mixture of complexes formed in the container is detected to monitor the treatment of the patient.

In still another embodiment, the invention provides methods for monitoring treatment of a patient in need thereof with a treatment ligand that binds specifically to the cell surface expressed target CD52. The CD52 treatment monitoring method includes obtaining a container containing a sample comprising a bodily fluid containing CD52⁺ cells obtained from the patient; incubating the sample in the container under conditions and for a time sufficient to allow complex formation with i) a first ligand that binds specifically to the expressed target at the binding site of the treatment ligand conjugated to a first distinguishable fluorescent label and ii) one assay component selected from a second ligand that binds the expressed target at a different binding site than the treatment ligand conjugated to a second distinguishable ligand; a third ligand that binds specifically to human immunoglobulin conjugated to a third distinguishable fluorescent label; and a first distinguishable capture particle linked to a CD52 antigen. Fluorescence from the fluorescent labels in the complexes formed in the container is detected substantially simultaneously to monitor the treatment of the patient.

In yet another embodiment, the invention provides methods for monitoring treatment of a patient in need thereof with a treatment ligand that binds specifically to the cell surface expressed target CD52. The CD52 treatment monitoring method can include incubating the following assay components in a container under conditions and for a time sufficient to allow complex formation between: 1) a sample comprising a bodily fluid containing CD52⁺ cells obtained from the patient; 2) a first distinguishable capture particle linked to a CD52 antigen; 3) a second distinguishable capture particle linked to the treatment ligand. Complexes formed by this first incubation are again incubated under conditions and for a time sufficient to allow binding interaction with the following additional assay components to form a mixture of complexes: 1) a first ligand that binds the expressed target at a different binding site than the treatment ligand and is conjugated to a first distinguishable fluorescent label; 2) a second ligand that binds specifically to the expressed target at the binding site of the treatment ligand and is conjugated to a second distinguishable fluorescent label; and 3) a third ligand that binds specifically to human immunoglobulin and is conjugated to a third distinguishable fluorescent label. The presence of fluorescence from the first, second or third fluorescent labels in the mixture of complexes formed in the container is detected substantially simultaneously to monitor the treatment of the patient.

In still another embodiment the invention provides methods for monitoring side effects of heparin therapy in a patient in need thereof that includes incubating the following assay components in a container under conditions and for a time sufficient to allow complex formation between: 1) a sample comprising stabilized whole blood of the patient; 2) a distinguishable capture particle linked to heparin:platelet factor 4 complex; 3) a first soluble ligand that binds specifically to a platelet activation antigen and is conjugated to a first fluorescent label; and 4) a second soluble ligand that binds specifically to platelets and is conjugated to a second fluorescent label. Complexes formed by this first incubation are again incubated in the container under conditions and for a time sufficient to allow binding interaction with a third soluble ligand that binds specifically to human immunoglobulins conjugated to a third fluorescent label to form a mixture of complexes. Fluorescence from the first fluorescent label, second fluorescent label or the third fluorescent label in the complexes formed in the container is detected substantially simultaneously to monitor heparin therapy in the patient.

In another embodiment, the invention provides kits for monitoring treatment of a patient with a treatment ligand that binds specifically to the cell surface expressed target CD20. In this embodiment, the kit includes a first soluble ligand that binds specifically to intracellular CD20; and one or more of the following: 1) a first soluble ligand that binds specifically to CD20⁺ cells; 2) a second soluble ligand that binds specifically to CD19⁺ cells; 3) capture particle linked to CD20 antigen; and 4) one to three distinguishable fluorescent labels for conjugation to the ligands.

In yet another embodiment, the invention provides kits for monitoring treatment of a patient with a treatment ligand that binds specifically to CD52 antigen. This CD52 monitoring kit includes a) a first distinguishable capture particle linked to a CD52 antigen; and one or more of the following: 1) a second distinguishable capture particle linked to the treatment ligand; 2) a first soluble ligand that binds the expressed target at a different binding site than the treatment ligand; 3) a second soluble ligand that binds specifically to the expressed target at the binding site of the treatment ligand; 4) a third soluble ligand that binds specifically to human immunoglobulin; and 5) three distinguishable fluorescent labels for conjugation to the ligands.

In still another embodiment, the invention provides kits for monitoring heparin therapy of a patient. The invention heparin monitoring kit includes a distinguishable capture particle linked to a heparin:platelet factor 4 complex; and one or more of the following: 1) a first soluble ligand that binds specifically to a platelet activation antigen; 2) a second soluble ligand that binds specifically to platelets; 3) a third soluble ligand that binds specifically to human immunoglobulins; and e) three distinguishable fluorescent labels for conjugation to the ligands.

### BRIEF DESCRIPTION OF THE DRAWINGS

FIG. 1 is a schematic diagram depicting the method of the present invention employing “sandwich assay” steps, in which the capture medium is coated with a ligand for the soluble target which is an antibody, a second soluble ligand (antibody) that binds to the cellular target and which is associated with a fluorescent label FL1, and a third soluble ligand (antibody) that binds the soluble target and is associated with a second fluorescent label FL2. The substantially simultaneous evaluation of the complexes formed is demonstrated by a flow cytometry graph of forward light scatter vs. log of side scatter where the solid phase capture medium can be gated separately from the cellular target(s) and two separate graphs of the number of fluorescent events associated with the gated population(s) can be determined. L=lymphocytes; M=monocytes; G=granulocytes.

FIG. 2A is a schematic diagram depicting the method of the present invention employing “competitive inhibition assay” steps. The capture medium is coated with soluble analyte. Also employed in the method are a soluble ligand for the cellular target that is an antibody and which is associated with a fluorescent label FL1, and a second soluble ligand (antibody) that binds to the analyte in the sample or on the bead, and which is associated with a fluorescent label FL2. The potential complexes formed are (1) a complex formed by the soluble ligand-FL1 and the cellular target, (2) a complex formed by the capture medium with immobilized soluble analyte and the second soluble ligand-FL2 (that has not bound to soluble analyte in the sample), and (3) the soluble analyte in the sample, if any, and the second soluble ligand-FL2. The substantially simultaneous evaluation of the complexes (1) and (2) formed is demonstrated by a flow cytometry graph of forward light scatter vs. log of side scatter where the solid phase capture medium are gated separately from the cellular target(s) and two separate graphs of the number of fluorescent events associated with the gated population(s) are shown. The measurement of the FL2 on the capture medium-immobilized analyte-ligand-FL2 complex is inversely proportional to the amount of soluble analyte in the sample due to competition for binding between the immobilized analyte and the soluble analyte in the sample. L=lymphocytes; M=monocytes; G=granulocytes.

FIG. 2B is schematic diagram depicting the method of the present invention employing alternative “competitive inhibition assay” steps. In this alternative, the capture medium is coated with the soluble ligand that binds the soluble analyte. Also employed in this method is a soluble ligand for the cellular target that is an antibody and which is associated with a fluorescent label FL1. Another component of the method is a soluble analyte that is associated with a fluorescent label FL2. The potential complexes formed are (1) a complex formed by the soluble ligand-FL1 and the cellular target, (2) a complex formed by the capture medium with immobilized ligand for the soluble analyte and the soluble analyte-FL2, and (3) a complex formed by the capture medium with immobilized ligand for the soluble analyte and the soluble analyte in the sample, if any. The substantially simultaneous evaluation of the complexes (1) and (2) formed is demonstrated by a flow cytometry graph of forward light scatter vs. log of side scatter where the solid phase capture medium is gated separately from the cellular target(s) and two separate graphs of the number of fluorescent events associated with the gated population(s) are shown. The measurement of the FL2 on the capture medium-immobilized ligand-bound analyte-FL2 complex is inversely proportional to the amount of unlabeled soluble analyte in the sample. L=lymphocytes; M=monocytes; G=granulocytes.

FIG. 3 is a schematic diagram depicting the method of the present invention employing “immune complex” steps. The bead is coated with streptavidin. A first ligand (antibody) to the cellular target is associated with a fluorescent label FL1. A second ligand (antibody) to the cellular target is associated with a fluorescent label FL2. A third ligand (antibody associated with biotin) is targeted to the soluble analyte. The substantially simultaneous evaluation of the complexes formed is demonstrated by a flow cytometry graph of forward light scatter vs. log of side scatter where the solid phase capture medium is gated separately from the cellular target(s) and two separate graphs of the number of fluorescent events associated with the gated population(s) is shown. L=lymphocytes; M=monocytes; G=granulocytes.

FIG. 4 is a schematic diagram depicting embodiments of the invention methods for monitoring a patient being treated with a ligand that binds specifically to a cell surface expressed target CD20. An antibody that binds specifically to the cell surface expressed target, CD20, is conjugated to a fluorescent label FL2 (clone HRC20)-PE). A second antibody that identifies the cell lineage (i.e. CD19) is conjugated to a second fluorescent label FL3 (CD19-ECD®). A capture medium is covalently linked to CD20 antigen. A third antibody that binds specifically to intracellular CD20 is conjugated to a third fluorescent label FL1 (clone L26-FITC). The substantially simultaneous evaluation of the complexes formed is demonstrated by a flow cytometry graph of forward light scatter vs. log of side scatter to differentiate the capture medium from the cells and a side scatter vs. CD19-ECD to gate the B cells separately from the remaining cells. These graphs can then be used to provide two graphs, one showing the number of events and mean intensity of fluorescent label FL2 on capture beads, and the other showing the percentages of B-cells containing CD20 on the surface (FL2) and intracellularly (FL1). L=lymphocytes; M=monocytes; G=granulocytes.

FIG. 5 is a schematic diagram depicting embodiments of the invention methods for monitoring of a patient being treated with a treatment ligand that binds specifically to a cell surface expressed target CD52. A first ligand (antibody) that binds specifically to expressed CD52 is conjugated to fluorescent label FL2 (CAMPATH 1G-PE) A second antibody that binds the same expressed target antigen at a different site is conjugated to second fluorescent label FL-5 (HI186)-PC7). A third antibody that binds specifically to human immunoglobulin (e.g. anti-HuIgG-FITC) is conjugated to a third fluorescent label FL1. One capture bead is covalently linked to synthetic CAMPATH antigen having no reactivity with HI186 and the other is covalently linked to the treatment antibody CAMPATH 1H. The substantially simultaneous evaluation of the complexes formed is demonstrated by a flow cytometry graph of forward light scatter vs. log of side scatter to differentiate the two types of capture medium and separate them from the cellular target. The lymphocyte cell population can then be gated into a graph of FL 2 vs. FL5 to assess the percentage of B-cells containing both epitopes of CD52. The CAMPATH antigen capture beads are gated into a Count vs. FL1 or FL2 histogram to show the number of beads containing circulating drug (CAMPATH-1H antibody) while the CAMPATH-1H (drug) capture bead can be gated into two separate histograms-one depicting the amount of autoantibody in the plasma (Count vs. FL1) and the other depicting the amount of shed CD52 antigen in the plasma (Count vs. FL2 or FL5) L=lymphocytes; M=monocytes; G=granulocytes.

FIG. 6 is a schematic diagram depicting embodiments of the invention methods as used to monitor treatment of a patient with heparin therapy for development of HIT. A bead is covalently linked to heparin:platelet factor 4 (H:PF4) complex (Bead-H:PF4). An antibody that binds specifically to a platelet activation antigen (e.g. CD62p-FITC) is conjugated with a first fluorescent label FL1. A second antibody that identifies platelets (i.e. CD41 is conjugated with a second fluorescent label, FL5 (e.g., CD41-PC7). A third antibody that binds specifically to anti-H:PF4 autoantibodies (e.g. anti-HuIg-PE) is conjugated with a second fluorescent label (FL2). The substantially simultaneous evaluation of the complexes formed is demonstrated by a flow cytometry graph of the log of forward light scatter vs. log of side scatter to differentiate the beads from the cells. The beads can be gated to a Count vs. FL2 histogram to measure the amount of circulating autoantibodies. The cells from the log FS vs. log SS histogram are gated into a histogram depicting FL5 vs. log FS to distinguish the red cells and white cells from the platelets. The platelets can then be sent to a FL2 vs. FL1 histogram to assess the proportion of activated platelets containing autoantibodies to H:PF4. Platelet concentration can be determined by the ratio of the red cells to the platelets or by comparison to the bead count. (PLT=platelets; RBC=red blood cells).

### DETAILED DESCRIPTION OF THE INVENTION

The method of the present invention answers the need in the art by providing for the substantially simultaneous evaluation (detection and/or measurement) of both soluble and bound targets in a sample. The method involves generally the analysis of a sample, which contains at least one target bound to a larger structure and at least one soluble analyte, which is unbound and free in the solution of the sample.

The general steps of the method involve adding to a single container the sample with (i) at least one soluble ligand that binds the cellular target, (ii) at least one soluble ligand that binds the soluble analyte or at least one competing soluble analyte that is preferably associated with a detectable label; and (iii) a solid phase capture medium that binds directly or indirectly to the soluble analyte or to the soluble ligand that binds the soluble analyte. After appropriately incubating the sample with these additives, the sample is substantially simultaneously analyzed without physically separating the different complexes that form within the sample. For example, one potential complex forms between the cellular target and at least one soluble ligand. Another potential complex forms between the capture medium bound directly to the soluble analyte (either labeled or unlabeled). Generally this direct binding involves a capture medium having immobilized thereon at least one ligand (e.g., a monoclonal antibody) that binds to the analyte. Still another complex may form between the capture medium bound indirectly to the soluble analyte. In this instance, the capture medium has coated thereon a ligand (e.g., biotin) that binds to another ligand (e.g., streptavidin) that is attached to a ligand for the soluble analyte (e.g., a monoclonal antibody that binds the analyte). Another complex may form between the capture medium bound to the soluble ligand that is bound to the soluble analyte. In this instance, the capture medium has coated thereon the soluble analyte, which binds the soluble ligand for the analyte.

It should be understood that one or more of the ligands employed in these methods are labeled with one or more detectable markers, as described in more detail below. In certain competitive inhibition assay formats, one or more soluble analytes employed in these methods are labeled with one or more detectable markers, as described in detail below. The lack of a physical separation step, i.e., the ability to measure the relevant complexes in the same container, in this method provides a valuable advantage in terms of efficiency and time in obtaining results of analysis, and further provides an advantage of preserving a small or rare sample, by using as little sample as possible.

The Sample

Preferably the sample is a biological sample, in which the bound target is a cell bearing at least one cellular target, and having at least one soluble analyte. The biological sample preferably contains cells of various types of biological tissue. For example, certain biological samples include, without limitation, whole blood, saliva, urine, synovial fluid, bone marrow, cerebrospinal fluid, vaginal mucus, cervical mucus, sputum, semen, amniotic fluid, cell lines, cell-containing exudates, cell-containing media, cell-containing buffer, bacterial samples, viral sample, and other exudates from a patient containing bacteria or virus. Such samples may further be diluted with saline, buffer or a physiologically acceptable diluent. Preferably such dilution occurs before addition of the soluble ligand(s) or of the competing soluble analyte(s).

In such biological sample, the cell type bearing the cellular target may be biological cells, particularly mammalian hematological or blood cells, and also all vertebrate or invertebrate cells, insect cells, bacterial cells, parasites, yeast or fungal cells, algal or other plant cells, etc. Also included in this definition are viruses, virus-like particles, parasites, and essentially any biological colloidal particle that has on its surface a receptor or antigen (i.e., an analyte) for which there exists a counter-receptor ligand or specific binding partner. The present invention is described specifically below using mammalian blood cells, specifically one or more of red blood cells and white blood cells. The white blood cells that may be present include, without limitation, granulocytes, monocytes/macrophages, platelets, lymphocytes, lymphoblasts, blast cells, leukocytes, and dendritic cells. Other cell types of cells used in these methods include, without limitation, fibroblasts, epithelial cells, epidermal cells, embryonic cells, hepatocytes, histiocytes, peritoneal cells, kidney cells, lung cells, sperm cells, oocytes, and normal and cancer cells of other mammalian tissue. The cellular target is generally, a cell surface antigen, an intracellular antigen, nuclear antigen, a fragment thereof, or a mixture of two or more of the preceding targets.

The soluble analyte, which is naturally occurring in such biological sample, or which is alternatively a competing soluble analyte employed as a component of certain embodiments of the methods of this invention, is likely to include, without limitation, a serum marker, a pharmaceutical drug, a protein, a virus, a hormone, a lipid, a nucleic acid sequence, a carbohydrate, a toxin, or an antigen shed from a cell type identified above, or produced or secreted by a mammalian cell, a bacterial cell, a virus, a cell infected by a virus, a cancer cell, a fungus, etc., or a fragment thereof, or a mixture of two or more of the preceding analytes.

Such naturally occurring targets and/or soluble analytes are desirable to detect or quantify due to their relationship to disease states. Thus detection of such targets is useful in diagnosis of disease, or monitoring of therapy, among others.

Still other types of sample which can be evaluated according to the method of this invention include water from any source, manufactured liquids such as gasoline, alcohol, pharmaceutical medicines, perfumes, food products, and the like. The targets and analytes in these samples may include adulterating compounds, such as drugs, poisons, toxins, microbial proteins and the like. Thus such targets are desirably detected as a means of quality control for detecting unwanted contamination or adulteration.

In certain embodiments, the sample can contain additional reagents. For example, where the sample is whole blood, the sample can contain an anti-coagulant, such as those described below. In another embodiment, the sample containing myeloid cells can contain an inhibitor of phagocytosis, such as discussed below. In still another embodiment, the sample can be treated with one or more of a fixative, a phosphatase inhibitor, or a calcium inhibitor.

Ligands Useful in the Invention

Generally, the components of the method include ligands that bind either the cellular target or the soluble analyte. By “ligand” is meant a moiety or binding partner that specifically binds to the target on the cell or to the soluble analyte. Such ligands are individually and independently an antibody that binds a cellular antigen, an antibody that binds a soluble antigen, an antigen that binds an antibody already bound to the cellular or soluble antigen; or fragments of such antibodies and antigens that are capable of binding; a nucleic acid sequence sufficiently complementary to a target nucleic acid sequence of the cellular target or soluble analyte to bind the target or analyte sequence, a nucleic acid sequence sufficiently complementary to a ligand nucleic acid sequence already bound to the cellular target or soluble analyte, or a chemical or proteinaceous compound, such as biotin or avidin.

The ligands can be soluble or can be immobilized on the capture medium (i.e., synthetically covalently linked to a bead), as indicated by the assay format. As defined herein, ligands include various agents that detect and react with one or more specific cellular targets or soluble analytes. Examples of ligands within the meaning of the present invention and their analytes include, without limitation, those listed in Table 1.

TABLE 1

| LIGAND | RECEPTOR |
|---|---|
| Antibody | Antigen |
| Natural ligands: cytokine or | CK receptor |
| chemokine | |
| Hormone | Hormone receptor |
| growth factor | Growth factor receptor |
| Secondary reagents: streptavidin | Biotinylated antibody - antigen |
| Antibody | Antibody - antigen |
| Synthetic peptides | Receptor |
| Solubilized natural ligands: | Receptor |
| Counter- receptor | |
| CTLA-4 | B7 (CD80/86) |
| Lectins (agglutinins) | Complementary carbohydrate or |
| | oligosaccharide on cell-surface |
| | Glycoprotein |
| MHC-peptide complex | T cell receptor (TCR) |
| Oligonucleotide | Complementary sequences in |
| | nucleic acids, DNA or RNA |

Those of skill in the art know methods useful for construction of such ligands. All such ligands are characterized by the desired ability to bind the specified target or analyte, whether it is soluble or bound to a cell. In one preferred embodiment, the ligand of the invention is a component that preferentially binds to all or a portion of a cell surface receptor. Thus, a ligand useful in this embodiment of the invention may be an antibody or a functional fragment thereof capable of binding to a cell surface receptor on a WBC population. Such antibodies or fragments include polyclonal antibodies from any native source, and native or recombinant monoclonal antibodies of classes IgG, IgM, IgA, IgD, and IgE, hybrid derivatives, and fragments of antibodies including Fab, Fab′ and F(ab′)2, humanized or human antibodies, recombinant or synthetic constructs containing the complementarity determining regions of an antibody, an Fc antibody fragment thereof, a single chain Fv antibody fragment, a synthetic antibody or chimeric antibody construct which shares sufficient CDRs to retain functionally equivalent binding characteristics of an antibody that binds a desired cell surface receptor, and a binding fragment produced by phage display.

Antibodies used in the examples of this invention were generally obtained by conventional hybridoma methods and purified from ascites fluid by ammonium sulfate (45%) precipitation, centrifugation and affinity chromatography using protein A. The standard process of making monoclonal antibodies is described in G. Kohler and C. Milstein, 1975 Nature, 256: 495-497. Of course, the particular method of making and the type of monoclonal antibody is not limited to such techniques and it is envisioned that any technique for making such antibodies is within the practice of the invention. Any ligand that can bind cellular targets or soluble analytes may be used, since the amplification of fluorescent intensities does not depend on the density of the particular receptor sites on a cell.

Other typical ligands can include, without limitation, a lectin, a hormone, a growth factor, or a synthetic peptide or chemical compound, or portions thereof that can bind the target or analyte. The selection of the ligand is not a limiting factor in this invention. Exemplary ligands are illustrated in the specific embodiments of methods described below and in the examples.

Detectable Labels or Markers

Where indicated, the ligands and/or the competing soluble analytes and/or the capture medium employed in the methods of this invention are associated (for example, linked covalently) with detectable labels or detectable markers. Detectable labels for attachment to components useful in this invention may be easily selected from among numerous compositions known and readily available to one skilled in the art of diagnostic assays. The reagents, ligands, competing analytes, or capture medium of this invention are not limited by the particular detectable label or label system employed. In some cases, the detectable “label” can include the refractive index of a cell surface or bead.

As used herein, the terms “label” or “marker” generally refers to a molecule, preferably proteinaceous, but also a small chemical molecule that is capable, acting alone, or in concert with other molecules or proteins, of providing a signal, that is detectable either directly or indirectly. In this invention, the marker is associated with the various ligands or competing analytes used in the assays. For example, a detectable label or marker can be a fluorescent label, a luminescent label, a radiolabel, or a chemiluminescent label linked (e.g, covalently) to an analyte, solid particle, cell, or ligand.

In one embodiment, preferred markers enable detection by emitting a detectable signal of a particular wavelength upon excitation by a laser. Phycobiliproteins, tandem dyes, certain fluorescent proteins, small chemical molecules, and certain molecules detectable by other means can all be considered markers for flow cytometry analyses. See, e.g., the markers listed in Handbook of Fluorescent Probes and Research Chemicals, 6th Ed., R. P. Haugland, Molecular Probes, Inc., Eugene, Oreg. (1996). “Phycobiliproteins” are a family of macromolecules found in red algae and blue-green algae. The biliproteins (the term “biliproteins” is equivalent to the term “phycobiliprotein”) have a molecular weight of at least about 30,000 daltons, more usually at least about 40,000 daltons, and may be as high as 60,000 or more daltons usually not exceeding about 300,000 daltons. The biliproteins will normally be comprised of from 2 to 3 different subunits, where the subunits may range from about 10,000 to about 60,000 molecular weight. The biliproteins are normally employed as obtained in their natural form from a wide variety of algae and cyanobacteria.

The presence of the protein in the biliproteins provides a wide range of functional groups for conjugation to proteinaceous and non-proteinaceous molecules. Functional groups that are present include amino, thiol, and carboxyl. In some instances, it may be desirable to introduce functional groups, particularly thiol groups when the biliprotein is to be conjugated to another protein. Each phycobiliprotein molecule contains a large number of chromophores. An exemplary ligand, e.g., an antibody molecule directly labeled with fluorescein will have between 1 and 3 chromophores associated with it. An antibody molecule (for example) directly labeled by conjugation with a phycobiliprotein may have as many as 34 associated chromophores, each with an absorbance and quantum yield roughly comparable to those of fluorescein.

Examples of phycobiliproteins useful in the present invention are phycocyanin, allophycocyanin (APC), allophycocyanin B, phycoerythrin (PE) and preferably R-phycoerythrin. PE is among the brightest fluorescent dyes currently available. Conjugated to an antibody, PE has been used to detect interleukin-4 in a fluorescent plate assay and found to be the only tested fluorescent label that produced adequate signal (M. C. Custer and M. T. Lotze, 1990 J. Immunol. Methods, 128, 109-117).

The tandem dyes are non-naturally occurring molecules that may be formed of a phycobiliprotein and another dye. See, for example, U.S. Pat. No. 4,542,104 and U.S. Pat. No. 5,272,257. Examples of tandem dyes useful in the present invention are phycoerythrocyanin or PC5 (PE-Cy5, phycoerythrin-cyanin 5.1; excitation, 486-580 nm, emission, 660-680 nm) [A. S. Waggoner et al, 1993 Ann. N.Y. Acad. Sci., 677:185-193 and U.S. Pat. No. 5,171,846] and ECD (phycoerythrin-texas red; excitation, 486-575 nm, emission, 610-635 nm) [U.S. Pat. No. 4,542,104 and U.S. Pat. No. 5,272,257. Other known tandem dyes are PE-Cy7, APC-Cy5, and APC-Cy7 μM. Roederer et al, 1996 Cytometry, 24:191-197]. Tandem dyes, PC5 and ECD, have been successfully directly conjugated to monoclonal antibodies by several methods that involve iminothiolane activation of the dye.

Preferably, the ligands and/or competing analytes and/or capture medium of this invention are associated with, or conjugated to fluorescent detectable fluorochromes, e.g., fluorescein isothiocyanate (FITC), phycoerythrin (PE), allophycocyanin (APC), or tandem dyes, PE-cyanin-5 (PC5), PE-cyanin-7 (PC7), and PE-Texas Red (ECD). The biliproteins and tandem dyes are commercially available from various sources including Beckman Coulter, Inc., Miami, Fla., Molecular Probes, Inc., Eugene, Oreg. and Prozyme, Inc., San Leandro, Calif. All of these fluorescent dyes are commercially available, and their uses known to the art.

Still other markers that may be directly conjugated to the components of the methods of this invention and used with the phycobiliproteins or tandem dyes in this invention to add additional numbers of markers (labeled ligands) to the method include small molecules that upon excitation emit wavelengths of less than 550 nm. Such molecules do not overlap with the emissions of the phycobiliproteins. One example of such a marker is fluorescein isothiocyanate (FITC). Others are listed in the Handbook cited above.

Still other markers that may be employed in this method to provide additional colors are the proteins known as the green fluorescent proteins and blue fluorescent proteins; also useful may be markers that emit upon excitation by ultraviolet light.

A marker can be an enzyme that interacts with a substrate to produce the detectable signal. Another marker embodiment can be a protein that is detectable by antibody binding or by binding to a suitably labeled ligand. A variety of enzyme systems operate to reveal a colorimetric signal in an assay, e.g., glucose oxidase (which uses glucose as a substrate) releases peroxide as a product that in the presence of peroxidase and a hydrogen donor such as tetramethyl benzidine (TMB) produces an oxidized TMB that is seen as a blue color. Other examples include horseradish peroxidase (HRP) or alkaline phosphatase (AP), and hexokinase in conjunction with glucose-6-phosphate dehydrogenase that reacts with ATP, glucose, and NAD+ to yield, among other products, NADH that is detected as increased absorbance at 340 nm wavelength.

Other label systems that may be utilized in the methods of this invention are detectable by other means, e.g., colored latex microparticles (Bangs Laboratories, Indiana) in which a dye is embedded may be used in place of enzymes to form conjugates with the inhibitor sequences or ligands and provide a visual signal indicative of the presence of the resulting complex in applicable assays. Still other label systems that may be used include nanoparticles or quantum dots.

In another embodiment such markers may preferably be reporter genes that upon expression produce detectable gene products. Such reporter sequences include without limitation, DNA sequences encoding a lux gene, beta-lactamase, a galactosidase enzyme, e.g., beta-galactosidase (LacZ), alkaline phosphatase, thymidine kinase, green fluorescent protein (GFP), chloramphenicol acetyltransferase (CAT), a luciferase enzyme, or a gluconase enzyme.

Still other suitable marker that may be attached to the components of the methods of this invention include membrane bound proteins including, for example, CD2, CD4, CD8, the influenza hemagglutinin protein, a biotin molecule, an avidin molecule, and others well known in the art, to which high affinity antibodies directed thereto exist or can be produced by conventional means. Another class of markers includes fusion proteins comprising a membrane bound protein appropriately fused to an antigen tag domain from, among others, hemagglutinin or a Myc gene. Still other detectable labels may include hybridization or PCR probes.

Any number of additional, and conventionally employed, marker systems may be adapted to the method of this invention. One of skill understands that selection and/or implementation of a label system involves only routine experimentation. The labels and markers discussed above may be obtained commercially from known sources.

Solid Phase Capture Medium

The solid phase capture medium is typically a capture particle, such as a physiologically compatible bead or a stabilized cellular particle, with any characteristic that allows it to be separated from the cell population of the sample. Such characteristics include refractive index, size, light scatter intensity (forward, side or 90°), or carrying a fluorescent detector dye to provide a unique fluorescent signature. Such beads suitable for use as capture particles in the invention methods are conventionally available in the art. For example, one subset of solid phase capture medium includes stable colloidal particles, such as polystyrene beads ranging in size from between about 0.2 to about 5.0 microns in diameter (i.e., colloidal-sized). Such polystyrene substrates or beads can contain aldehyde and/or sulfate functional groups, such as the commercially available beads, e.g., from Interfacial Dynamics Corporation, Portland, Oreg.

Alternatively, the polystyrene bead has an aminodextran coating over its peripheral surface and/or a colloidal-metal coating. Preferably an aminodextran coating is covalently bonded to the core substrate by covalent bonds between the free amino groups of the aminodextran and the amine-reactive functional groups of the polystyrene substrate and further by crosslinking with an agent such as glutaraldehyde. The aminodextran coating may generally be characterized as having a degree of diamine substitution in the range of 1/40- 1/35 (1×-aminodextran) compared to a maximum theoretical value of 1/2.5. More preferably, the diamine substitution in the aminodextran coating is approximately 1/7 to ⅛ (5×-aminodextran). Analytes, particularly protein analytes, may be readily attached to these beads as is taught in the references cited below. See also, O. Siiman et al, “Covalently Bound Antibody on Polystyrene Latex Beads: Formation, Stability and Use in Analyses of White Blood Cell Populations”, J Colloid Interface Sci., 233: (January 2001).

A variety of aminodextran beads are described in U.S. Pat. Nos. 6,074,884; 5,945,293; and 5,658,741. Aminodextran-coated monodispersed colloidal dispersions of magnetic ferrite [U.S. Pat. No. 5,240,640], metal [U.S. Pat. No. 5,248,772], polystyrene [U.S. Pat. No. 5,466,609; U.S. Pat. No. 5,707,877; U.S. Pat. No. 5,639,620; U.S. Pat. No. 5,776,706], and polystyrene-metal [U.S. Pat. No. 5,552,086; U.S. Pat. No. 5,527,713] particles may also be employed as formed bodies according to this invention.

Another type of bead may contain the above-described coated substrate with a layer of colloidal-sized metallic solid overlaying the aminodextran coating. Preferably this layer is uniformly dispersed over the dispersed surface of the aminodextran layer. The colloidal metal useful in forming the coated substrate is generally described as a metal which can be reduced from the ionic state to the metal(0) state by the aminodextran coating, or a metal which can form metal ions or metal ion complexes which have a reduction potential of about +0.7 volts or higher. While such metal ions may include: Ag(I), Au(III), Pd(II), Pt(II), Rh(III), Ir(III), Ru(II), Os(II), the preferred metal ions for such use are colloidal gold(III) and colloidal silver(I). Specifically, gold/silver colloid coated polystyrene-aminodextran beads, their preparation, characterization and use in analyses of subpopulations of white blood cells in whole blood have been described. See, e.g., U.S. Pat. No. 5,248,772; U.S. Pat. No. 5,552,086; U.S. Pat. No. 5,945,293; and O. Siiman and A. Burshteyn, 2000 J. Phys. Chem., 104:9795-9810; and O. Siiman et al, 2000 Cytometry, 41:298-307.

An alternative to this coated bead employs carboxy-functionalized polystyrene particles as the core substrate, coated with aminodextran by EDAC coupling as described in U.S. Pat. No. 5,639,620.

Other suitable beads that may be utilized in the methods of this invention are colored latex microparticles (Bangs Laboratories, Indiana) in which a dye is embedded and may be used to form complexes with the target, analyte or ligands. These beads also provide a visual signal indicative of the presence of the resulting complex in applicable assays. Still other suitable beads include nanocrystals, quantum dots and similar materials.

In one embodiment the bead is from 0.05 to 20 microns in diameter. In another embodiment, the bead is from 5 to 7 microns. In still another embodiment, the capture medium is greater than 1 μM in size. Mixtures of a variety of sizes of beads may also be employed, particularly where there are more than one soluble analyte to be detected. Generally, bead size impacts the sensitivity range of the assay, because smaller beads bind fewer antibodies (see e.g., Lindmo, cited above or Frengen cited above). Therefore, in one embodiment, in which high sensitivity is required, a smaller number of larger beads is desirable for the assays. In the presence of large numbers of soluble analytes, a higher number of beads (both large and small) may be employed in these methods. For use in some embodiments of the present invention, the capture medium or bead is larger than the soluble analyte to be detected.

The capture medium may have bound thereto multiple ligands or multiple competing analytes. Each ligand bound to the capture medium is capable of binding to a soluble analyte or binding to an antibody that is itself capable of binding to the soluble analyte. Each competing analyte bound to the capture medium is capable of binding to a ligand (e.g., an antibody) that is capable of binding to the soluble analyte (whether labeled or unlabeled). Such ligands or competing analytes are associated or immobilized on the capture medium by conventional methods. For example, ligands or analytes such as antibodies, antigens, or linkers (e.g. Streptavidin, Protein A) may be attached to beads depending upon format of the analyte assay (competitive, immune-complex or sandwich) as described below. The beads may also be associated with detectable labels, preferably fluorescent labels, such as discussed above. Methods for attachment with such labels are disclosed in the texts cited herein.

Beads may be fluorescent or non-fluorescent, may be of different sizes or different fluorescent intensities, or both, for differentiation of multiple analytes. If using fluorescent intensity for labeling beads, it is preferred that the fluorescence emission should be unique for each population directed to a different analyte. Bead populations of different intensity are preferably resolvable if fluorescence of the bead is used as the only detectable label for discriminating among the soluble analyte and cellular target. Alternatively, if the size of the bead populations is used as the sole detectable label for discrimination among the soluble analyte and cellular target, each bead population must have a different forward scatter (FS) or side scatter (SS) than the cell population of interest in the assay.

Where the resulting analytic steps involve flow cytometry, the minimal parameters or characteristics of the beads are scatter (forward scatter (FS) and/or side scatter (SS)) or at least two fluorescent wavelengths.

The relative volumes of the bead used in the sample container of the methods described herein are dependent upon bead concentration, analyte detection limits, and the cellular target, and sample size. For example, in one embodiment about 10 μL beads may be added to per 50-100 μL blood for 12×75 test tubes vs. 5 μL beads for 25-50 μL blood for microplate assays.

Preferably and optionally, solutions of bead populations useful in the present invention include a reagent that inhibits phagocytosis of the capture medium without damaging the target cells or inhibiting binding the target cells and the ligands.

Additionally or alternatively, the bead solution may contain an anti-coagulant, such as those mentioned below. Further the bead solutions may be kept at a temperature below 37° C., and more preferably, below 25° C., prior to addition to the sample or when introduced into the sample. These alternative and optional steps are also useful for inhibiting phagocytosis of the beads when in the sample.

Assay Formats

This method may utilize any number of conventional assay formats, for example, sandwich assays, competitive inhibition assays, immune complex assays, or others. Some of the components of these assays, as well as the conditions under which the sample is incubated, and the inclusion of optional steps or reagents, are dependent upon the assay selected. However, surprisingly, these assays using both beads and cellular markers provide accurate results in a single analysis. There is no negative effect on the binding of the beads in the presence of the cellular markers or vice versa. Surprisingly, there is no effect on the measurement of light scatter of fluorescent properties of the cells in the presence of the beads.

### (1) Sandwich Assay

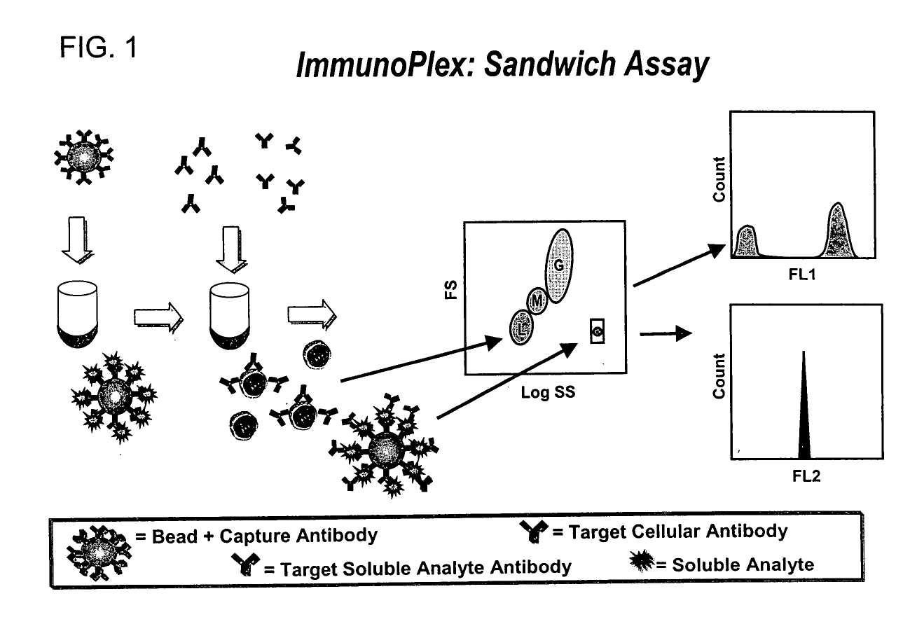

In one embodiment, the method can include the following steps for a sandwich assay. See, e.g., FIG. 1. A sample is introduced into a container, such as a microtiter plate wells or test tubes. The solid phase capture medium is added to the container. In this assay, the capture medium or bead has immobilized thereon multiple first ligands that are capable of binding the soluble analyte. Preferably the method employs mixing, and incubation at a temperature of 37° C. or lower for about 5 minutes to up to 3 hours, and preferably for about 60 minutes. In one embodiment, the temperature is desirable lower than 25° C. or 22° C. The temperature and incubation times can be selected by one of skill in the art based upon the analyte, the analyte detection limit and the identity of the cellular target in the sample.

Thus after incubation and occasional mixing, a “first” complex is formed in the sample which consists of the capture medium, multiple immobilized first ligands, and multiple soluble analyte now bound to the capture medium by the first ligands.

An optional washing step may be employed before addition of the following components, depending upon required assay sensitivity. In some embodiments of these methods, a wash step to eliminate unbound first ligands is required for increased sensitivity.

Thereafter, suitable concentrations of at least two additional ligands are added to the sample. One of the additional ligands is a soluble second ligand smaller than the cellular target. The second ligand is capable of binding to the cellular target, e.g., to a cell surface or intracellular moiety. For example, an antibody to the cell surface antigen is a suitable ligand here. Each second ligand is desirably associated with a detectable label such as described above, and multiple second ligands can bind to a single target cell. The other of the additional ligands is a third ligand that is capable of binding to the soluble analyte whether that analyte be immobilized on the capture medium in the first complex or remaining soluble in the sample. This third ligand is desirably associated with a second detectable label that is different from the detectable label of the second ligand, i.e., the ligand that binds the cellular target.

In certain embodiments of this sandwich assay method, there are more than one second ligand directed to more than one target on the same cell type (e.g., an anti-CD45-PC5 antibody to the cell surface antigen CD45 and an anti-CD 14-FITC antibody to the cell surface antigen CD14). In still other embodiments, more than one soluble ligand is directed to the same or to different targets on the same or different cell types. In certain embodiments of this assay more than one ligand is employed to more than one soluble analyte (e.g., an anti-IL-2-PE antibody to the soluble analyte IL-2, an anti-IL-6-PC7 antibody to the soluble analyte IL-6). Alternatively, more than one soluble ligand may be used for the same soluble analyte, or the same fluorochrome may be used for more than one soluble analyte.

After these components are added to the sample, the sample is mixed, and incubated with occasional mixing as described above. Thus the sample now contains a second complex consisting of the second labeled ligand(s) bound now to the cellular target(s) and a third complex comprising the third ligand bound to the soluble analyte which is bound through the first ligand to the capture medium. There may also be some small soluble third ligand-soluble analyte complexes in the sample.

Another optional step may be inserted into the assay method at this point, if the sample contains non-nucleated cells, such as red blood cells, and if higher sensitivity is needed for the analysis steps below. The sample may optionally be treated with an agent to lyse the non-nucleated cells. Another optional wash step may also be included to remove the lysed materials from the complexes or to remove excess unbound labeled ligands, depending upon required assay sensitivity.

The final step of this method is a substantially simultaneous analysis of the sample treated as described above, without physically separating the various complexes to be measured. Given the above steps of this method, one may take the sample containing these complexes and discriminate between the third complex comprising the third ligand bound to the soluble analyte which is bound through the first ligand to the capture medium and the second complex consisting of the second labeled ligand(s) bound now to the cellular target(s) using the same sample. Methods suitable for performing this analysis step include image analysis and, preferably, flow cytometric analysis. A flow cytometric analysis is conducted by employing a gating strategy appropriate to the sample type. For example, the third complex containing the beads is gated separately from the second complex of the ligand-labeled cells based on light scatter and/or fluorescence intensity. Thereafter, if more than one fluorescent label is present on the cell target or the bead, the strategy can provide separate compensation for each fluorescent label. Similarly other cell parameters, such as differentially expressed targets and intracellular targets may also be measured during this analysis. The amount of third complex detected is proportional to the amount of soluble analyte (unlabeled) present in the sample.

The standards for quantitation of the analyte include cell controls with serum-based analyte standards. Such standards are applicable to all assay types described herein. These standards are stabilized cells in a media containing the soluble analytes of interest.

### (2) Competitive Inhibition Assay

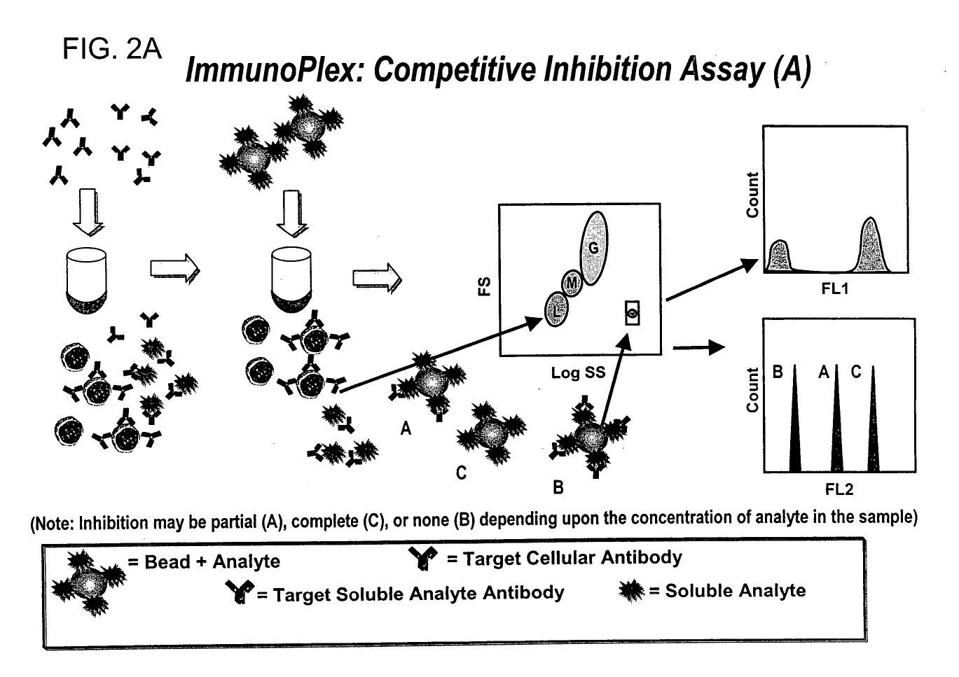

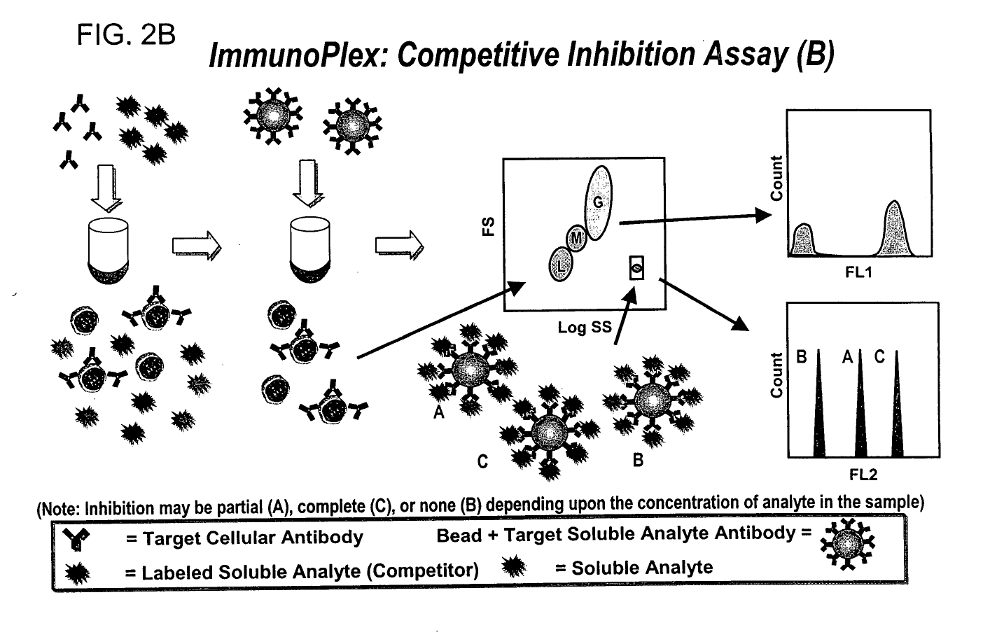

In still other embodiments, the method can include the following steps for a competitive inhibition assay. See, e.g., FIG. 2A and FIG. 2B.

In one format depicted in FIG. 2A, a sample is introduced into a container, such as a microtiter plate well or test tube. A known concentration of a first soluble ligand capable of binding to a single cellular target is added to the sample. This first ligand is desirably associated with a first detectable label. Multiple of the first ligands may bind to the cell. At the same time a known concentration of a second ligand capable of binding the soluble analyte is added to the sample. The second ligand is associated with a second detectable label. After mixing and incubating for from about 5 minutes to about 3 hours, preferably up to 60 minutes, at a temperature of under 37° C., a first complex is formed which includes the cellular target bound to the first labeled ligand and a second complex is formed comprising soluble analyte bound to the second labeled ligand.

Thereafter a solid phase capture medium on which are immobilized a known multiple of the same analytes is added to the sample. The sample is vortexed and incubated again under the same conditions, and a third complex is formed consisting of the capture medium, the analyte immobilized thereof and any of the second ligand in the sample that did not bind to the soluble analyte.

An optional washing step may be employed after the addition of the components, depending upon required assay sensitivity.

Another optional step may be inserted into the assay method at this point, if the sample contains non-nucleated cells, such as red blood cells, and if higher sensitivity is needed for the analysis steps below. The sample may optionally be treated with an agent to lyse the non-nucleated cells. Among such agents are included without limitation, ImmunoPrep™ reagents (Beckman Coulter), ammonium chloride, etc. Another optional wash step may also be included to remove the lysed materials from the complexes or to remove excess unbound labeled ligands, depending upon required assay sensitivity.

The final step of this method is a substantially simultaneous analysis of the sample treated as described above, without physically separating the various complexes to be measured. Given the above steps of this method, one may take the sample containing these complexes and discriminate between the first complex comprising the cellular target bound to the first labeled ligand and the third complex consisting of the capture medium, the analyte immobilized thereof and any of the second ligand in the sample that did not bind to the soluble analyte. The amount of third complex detected is proportional to the amount of soluble analyte present in the sample.

In one format depicted in FIG. 2B, a sample is introduced into a container, such as a microtiter plate wells or test tubes. A known concentration of a first soluble ligand capable of binding to a single cellular target is added to the sample. This first ligand is desirably associated with a first detectable label. Multiple of the first ligands may bind to the cell. At the same time a known concentration of a competing soluble analyte is added to the sample. The competing soluble analyte is preferably associated with a second detectable label. After mixing and incubating for from about 5 minutes to about 3 hours, preferably up to 60 minutes, at a temperature of under 37° C., a first complex is formed which includes the cellular target bound to the first labeled ligand.

Thereafter a solid phase capture medium on which are immobilized a known multiple of a ligand that binds to the soluble analyte (competing analyte or naturally occurring analyte in the sample, if any) is added to the sample. The sample is mixed and incubated again under the same conditions, and potential second and third complexes are formed. A second complex is formed by the capture medium-immobilized ligand and the naturally occurring soluble analyte in the sample, if any (unlabeled). A third complex is formed by any of the capture medium-immobilized ligand that did not bind to the unlabeled soluble analyte and the competing analyte (labeled). No complex is formed between the competing, labeled soluble analyte and the unlabeled soluble analyte occurring naturally in the sample.

An optional washing step may be employed after the addition of the components, depending upon required assay sensitivity.

Another optional step may be inserted into the assay method at this point, if the sample contains non-nucleated cells, such as red blood cells, and if higher sensitivity is needed for the analysis steps below. The sample may optionally be treated with an agent to lyse the non-nucleated cells. Among such agents are included without limitation, ImmunoPrep™ reagents (Beckman Coulter), ammonium chloride, etc. Another optional wash step may also be included to remove the lysed materials from the complexes or to remove excess unbound labeled ligands, depending upon required assay sensitivity.

The final step of this method is a substantially simultaneous analysis of the sample treated as described above, without physically separating the various complexes to be measured. Given the above steps of this method, one may take the sample containing these complexes and discriminate between the first complex comprising the cellular target bound to the first labeled ligand and the third complex consisting of the capture medium-immobilized ligand and competing analyte (labeled). Additionally the second complex of the capture medium with the unlabeled analyte may also be detected. The amount of third complex detected is proportional to the amount of soluble analyte (unlabeled) present in the sample.

As with the sandwich assay, one may manipulate this assay for measurement of more than one cell type, more than one cellular target on a cell type, or more than one soluble analyte by selecting from among any number of soluble ligands, detectable labels, and solid phase capture media on which is immobilized different ligands or competing analytes. Methods suitable for performing the analysis step include image analysis and, preferably, flow cytometric analysis. A flow cytometric analysis is conducted by employing a gating strategy appropriate to the sample type. For example, the complexes containing the beads are gated separately from the complex of the ligand-labeled cells based on light scatter and/or fluorescence intensity. Thereafter, if more than one fluorescent label is present on the cell target or the bead, the strategy can provide separate compensation for each fluorescent label. Similarly other cell parameters, such as differential and intracellular antigens or other targets may also be measured during this analysis.

The standards for quantitation of the analyte include cell controls with serum-based analyte standards. Such standards are applicable to all assay types described herein. These standards are stabilized cells in a media containing the soluble analytes of interest.

### (3) Immune Complex Assay

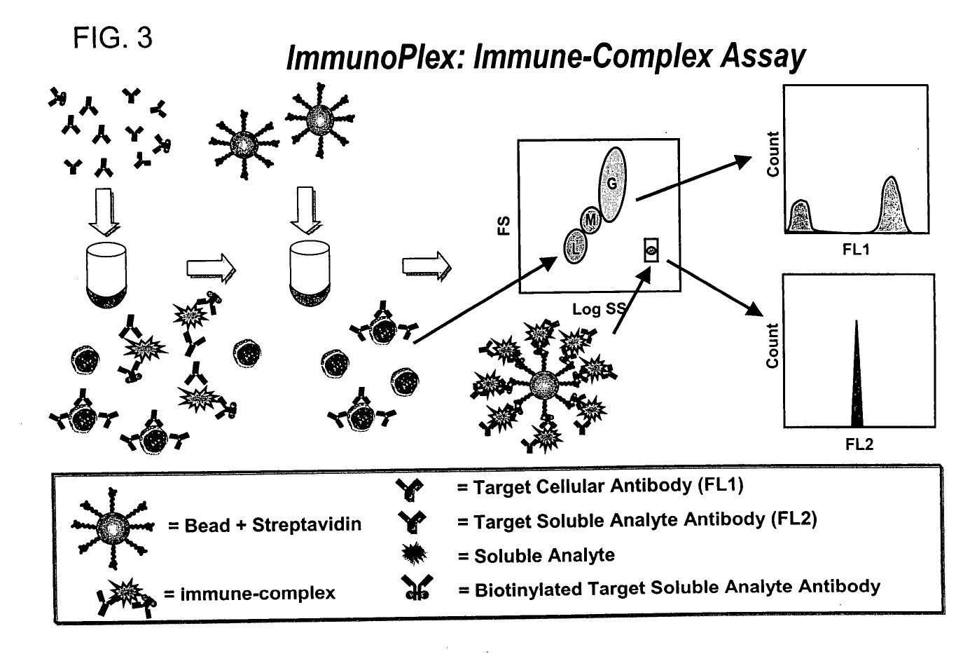

In one embodiment, the method can include the following steps for an immune complex assay. See, e.g., FIG. 3. A sample is introduced into a container, such as a microtiter plate well or test tube. A first soluble ligand capable of binding to the cellular target is added to the sample. Multiple of these first ligands may bind a single target cell. Desirably these first ligands provide a first detectable signal, preferably due to association with a detectable label. Also added to the sample is a second ligand, capable of binding to the soluble analyte. This second ligand is also preferably labeled and can provide a second detectable signal. To the same sample is added a third ligand capable of binding to the same soluble analyte, which ligand is associated with a different detectable label. After mixing and incubating for from about 5 minutes to about 3 hours, preferably up to 60 minutes, at a temperature of under 37° C., a first complex is formed comprising the first cellular target and the first ligand and a second complex is formed comprising the soluble analyte bound to one or both of the second ligand and third ligand.

Thereafter, a solid phase capture medium on which is immobilized multiple fourth ligands is added to the sample. These fourth ligands are capable of binding to the second or third ligands. After mixing, and incubating under the conditions described above, a third complex is formed. This third complex consists of the solid phase capture medium bound to multiple fourth ligands, with each fourth ligand bound to a third ligand. Each third ligand is also bound to a soluble analyte, which is then further bound to one or more second ligands.

An optional washing step may be employed after the addition of the assay components, depending upon required assay sensitivity. Another optional step may be inserted into the assay method at this point, if the sample contains non-nucleated cells, such as red blood cells, and if higher sensitivity is needed for the analysis steps below. The sample may optionally be treated with an agent to lyse the non-nucleated cells. Among such agents are included without limitation, ImmunoPrep reagents (Beckman Coulter), ammonium chloride, etc. Another optional wash step may also be included to remove the lysed materials from the complexes or to remove excess unbound labeled ligands, depending upon required assay sensitivity.

The final step of this method is a substantially simultaneous analysis of the sample treated as described above, without physically separating the various complexes to be measured. Given the above steps of this method, one may take the sample containing these complexes and discriminate between the first complex, the second complex and the third complex. The amount of third complex detected is proportional to the amount of soluble analyte (unlabeled) present in the sample.

As with the sandwich assay, one may manipulate this assay for measurement of more than one cell type, more than one cellular target on a cell type, or more than one soluble analyte by selecting from among any number of soluble ligands, detectable labels, and solid phase capture media on which is immobilized different analytes. Methods suitable for performing the analysis step include image analysis and, preferably, flow cytometric analysis. A flow cytometric analysis is conducted by employing a gating strategy appropriate to the sample type. For example, the complex containing the beads is gated separately from the complex of the ligand-labeled cells based on light scatter and fluorescence intensity. Thereafter, if more than one fluorescent label is present on the cell target or the bead, the strategy can provide separate compensation for each fluorescent label. Similarly other cell parameters, such as differential and intracellular targets or antigens may also be measured during this analysis.

The standards for quantitation of the analyte include cell controls with serum-based analyte standards. Such standards are applicable to all assay types described herein. These standards are stabilized cells in a media containing the soluble analytes of interest.

Optional Method Steps

The methods of this invention can also include a number of optional steps.

### (1) Washing Steps

For example, where increased sensitivity of the assays are desirable, washing steps with buffer, or diluent can be introduced into the methods. Generally, such washing steps can be introduced after the incubation of the sample with the capture medium to eliminate materials not bound to the capture medium. Alternatively, such washing steps can follow incubation with soluble ligand to eliminate uncomplexed materials. Still another option includes washing the sample after an optional lysis step to rid the sample of lysed red blood cell components.

### (2) Inhibiting Phagocytosis

Another optional step suitable for the methods of this invention is the addition of a reagent that inhibits phagocytosis of the capture medium by cells, particularly myeloid cells in the sample, without damaging the target cells or inhibiting binding the target cells and the ligands used in the methods. A suitable phagocytosis inhibitor is sodium azide (preferably, at a concentration of less than 0.01% v/v). Gliotoxin, gliotoxin-trisulfide and gliotoxin-tetrasulfide and related compounds belonging to the class of epipolythiodioxopiperazines also inhibit phagocytosis by macrophages, white cells that participate in the host's defense system. See, e.g., U.S. Pat. No. 4,886,796. Another suitable phagocytosis inhibitor is cytochalasin B (see also, U.S. Pat. No. 5,162,990). Other phagocytosis inhibitors include protein kinase inhibitors, an excess of heavy metals such as zinc, cadmium, lead, mercury, etc., phosphatase inhibitors such as pyrophosphate and levamisole, an excess of adenosine or the polyamines putrescine and spermidine, cycloheximide, EDTA, bromoenol lactone, and other phospholipase inhibitors and cytochalasin D.

The phagocytosis inhibitors may be added to the bead solutions particularly when the biological samples contain myeloid cells, because phagocytosis of beads by myeloid cells is common. While the phagocytosis-inhibiting reagent may be added to the capture medium prior to addition of the capture medium to the sample, it is also possible to add the phagocytosis-inhibiting reagent to the capture medium at the same time the capture medium is added to the sample. Alternatively, the phagocytosis-inhibiting reagent is added to the sample prior to addition of the capture medium to the sample. The phagocytosis-inhibiting reagent is added to the sample at the same time the capture medium is added to the sample. In one embodiment, it has been determined that where the method employs beads 1 μm or less in diameter, it is preferable to introduce multiple phagocytic inhibitors, such as combinations of the inhibitors identified above.

### (3) Lysis

Another optional step of the method for samples that contain blood cells includes lysing the sample to remove the generally very numerous non-nucleated blood cells, including red blood cells prior to the analyzing step. Lysing agents, preferably detergents, more preferably nonionic detergents, are used to break down cell membranes, thus releasing DNA, RNA and proteins from the cells. Any suitable lysing agent may be employed. Buffered halides, such as ammonium chloride and Trizma based (e.g., about 7.5 g ammonium chloride and 2 g Tris per liter), define one suitable class of lysing agents, where the undesired cells include red blood cells. Optionally, before the lysing, the cells are subjected to a preliminary fixing step, such as by contacting them with a suitable fixing agent, heating them or both. For instance, the cells are contacted with a buffered antimicrobial saline solution including a suitable amount of a fixative (e.g., about 0.11% formaldehyde).

Still other lytic agents are included without limitation, ImmunoPrep™ reagents (Beckman Coulter), ammonium chloride, etc. In one embodiment, a lytic reagent is Bacterial Protein Extraction Reagent (BPER), a proprietary mixture of nonionic detergents marketed by the Pierce Chemical Company. Other nonionic detergents are useful and many detergents are operable, even some anionic and cationic detergents under certain applications. The nonionic detergent lysing agents are generally be added to the sample in a concentration of about 0.1 to 5, more preferably 0.5 to 2 wt %. Other known lysing agents can also be used with the technology such as freeze/thawing, French cell press, enzymes, microfluidization, sonication, etc.

As stated above, the sample may then be optionally washed after lysis.

### (4) Adding an Inhibitor of Cellular Activation

Still another optional step that can be included in the methods described herein includes contacting the sample with an inhibitor of cellular activation. The inhibitor of cellular activation is contacted with the sample prior to or substantially simultaneously with the addition to the sample of the capture medium or ligands used in the methods. The inhibitor of cellular activation can be one or more of an anticoagulant, an inhibiting reagent, a fixative or an inhibiting reaction temperature.

### (a) Anticoagulants

Anticoagulation of the sample can be accomplished by binding or chelation of calcium ions by a variety of substances. Conventional anticoagulants include, without limitation, ethylenediaminetetraacetic acid (EDTA) or a salt thereof, a citrate salt of sodium or potassium, an oxalate salt of sodium or potassium, or combinations thereof. Other traditional anticoagulants include natural enzymatic inhibitors of the coagulation sequence, such as heparin or sodium fluoride or hirudin. Still other anticoagulants include, without limitation, protease, protein kinase inhibitors such as phenylmethylsulfonylfluoride (PMSF), 4-(2-aminoethyl) benzenesulfonyl-fluoride (AEBSF), tosyl-lysine chloro-methyl ketone (TLCK), tosyl-phenylalanine chloromethyl ketone (TPCK), leupeptin, epstatin A, 1-(5-isoquinolinesulfonyl) piperazine. Such anticoagulants or preservatives may be used alone or in combination for addition to the sample. See, for example, U.S. Pat. Nos. 5,935,857 and 4,528,274. Anticoagulants may be added to the sample in this invention preferably prior to the addition of the ligands and/or capture medium.

### (b) Fixatives

Another optional step to be added to the methods above includes the addition of a fixative to the sample prior to the introduction of the ligands or capture medium. “Fixatives” include formaldehyde, paraformaldehyde, and glutaraldehyde, dehydrating alcohols, glyoxal, and organic acids, such as acetic acid, formic acid, and picric acid, mercuric compounds, tannic acid and many other compounds. Another useful fixative is described in U.S. Pat. No. 5,459,073 which fixative has low toxicity employing a formaldehyde donor, such as diazolidinyl urea, imidazolidinyl urea, dimethylol-5,5-dimethylhydantoin, dimethylol urea and the like rather than formaldehyde itself.

### (c) Inhibiting Reagents

Still another optional step is to add to the sample an inhibiting reagent to control cellular activation. Suitable reagent compositions can include one or more protease inhibitor(s). A non-exclusive list of protease inhibitors for use in the present invention includes the serine protease inhibitors, such as 4-(2-aminoethyl)benzenesulfonyl fluoride hydrochloride (AEBSF), which has a molecular weight of 230.7 and inhibits catalytic activity of the protease active site; antithrombin plasma protein (60,000 MW) that inhibits thrombin and other serine proteases in the blood clotting cascade; or 4-amidinophenylmethanesulfonyl-fluoride-HCl (APMSF, 352.7 MW), an irreversible inhibitor of trypsin-like serine proteases. Still other serine proteases include Aprotinin (6500 MW) that inhibits serine proteases by tightly binding to the active site of the enzyme; diisopropyl phosphorofluoridate (DFP, 184.2 MW), a very toxic, irreversible inhibitors of serine proteases and acetylcholine esterase; phenylmethanesulfonyl fluoride (PMSF, 174.2 MW), which is another toxic, irreversible inhibitor that acts by chemically modifying the active site of the enzyme; and α-toluenesulfonyl fluoride.

Other suitable serine and cysteine protease inhibitors useful in the methods of this invention include antipain (678.2 MW), a reversible inhibitor of proteases and of RNA synthesis; chymostatin (600 MW), a reversible inhibitor of some serine and cysteine proteases; leupeptin (475.6 MW) a reversible competitive inhibitor of trypsin-like proteases; L-1-chloro-3-[4-tosyl-amido]-7-amino-2-heptanone-HCl (TLCK, 369.3 MW), which inhibits irreversibly by chemically altering the enzyme active site; and L-1-chloro-3-[4-tosylamido]-4-phenyl-2-butanone (TPCK, 351.8), which irreversibly inhibits by chemically altering the enzyme active site.

Still other suitable cysteine protease inhibitors useful in this invention include E-64 (357.4 MW), a non-competitive irreversible inhibitor of cystein proteases. Other suitable protease inhibitors inhibit metalloproteases. For example, amastatin (511 MW) is a non-toxic reversible inhibitor; bestsatin (244.8 MW) is a multi-function metallo-protease inhibitor that has anticarcinogenic and immunomodulating properties; diprotin (341.5 MW), a reversible inhibitor; EDTA (372.3 MW) a reversible inhibitor that acts by chelating enzyme cofactors and may interfere with other metal dependent biological processes. Other metaloprotease inhibitors include vanadium, molybdate salts, and 1,10-phenanthroline. Still other suitable inhibitors for use in this invention are aspartic protease inhibitors, such as pepstatin (685.9 MW) a peptide that provides reversible inhibition.

In one embodiment, the methods above include the step of introducing into the sample a single inhibitor. In another embodiment, the invention includes adding combinations of two or more such inhibitors, to permit use of small amounts of those inhibitors that are toxic or cause otherwise undesirable effects if used alone in large concentrations. It is desirable for the concentration of protease inhibitor(s) in the stabilizing reagent composition to be up to about 10 mM. However, the range of concentrations is entirely dependent upon the inhibitor(s) used. This range is determined based upon the experimental data of inhibition of platelet activation, as described herein. One of skill in the art given the teachings provided herein would readily be able to determine, with only a minimal and conventional amount of experimentation, a desirable concentration for each specific inhibitor used in the assay methods.

Another group of useful inhibitors includes one or more phosphatase inhibitor(s). A non-exclusive list of suitable phosphatase inhibitors includes, without limitation, pyrophosphate, microcystin 1A, microcystin 1R, tetramisole, 1-4-bromotetramisole, tautomycin, okadaic acid, calyculin, thrysiferyl-23-acetate, cantharidine, vanadium salts, sodium orthovanadate, tartrate salts, phloridzin, molybdate salts, and imidazole. For other suitable inhibitors, see Handbook of Enzyme Inhibitors, Melmward Sollner (1989), ISBN 3-527-26994-0; ISBN 0-89537-860-0, incorporated by reference herein. In one embodiment, the methods of this invention include adding a single phosphatase inhibitor to the sample.

In another embodiment, the methods of this invention include adding combinations of two or more such inhibitors, to permit use of small amounts of those inhibitors that are toxic or cause otherwise undesirable effects if used alone in large concentrations. It is desirable for the concentration of a phosphatase inhibitor(s) in the sample to be up to about 120 mM. However, the range of concentrations is entirely dependent upon the inhibitor(s) used. This range is determined based upon the experimental data of inhibition of platelet activation, as described herein. One of skill in the art given the teachings provided herein would readily be able to determine, with only a minimal and conventional amount of experimentation, a desirable concentration for each sample.

### (d) Temperature

Another optional step of the present invention useful for inhibiting cellular activation and making the processes of this invention more efficient is the use of inhibiting reaction temperatures in the method of below 25° C. Preferably, such lower temperature incubations can occur at a temperature of between 4° C. and 25° C. In one embodiment, the temperature is below 20° C. In another embodiment, the temperature is below 15° C. In still another embodiment, the temperature is below 10° C. In still another embodiment, the temperature is below 7° C. One of skill in the art given the disclosures herein may readily select the appropriate temperature for the method employed.

Specific Methods of the Invention

The methods of the present invention are useful in diagnosis of a variety of mammalian diseases or conditions. Examples of such diseases or conditions include, without limitation, sepsis, inflammation, autoimmune disease, cardiovascular disease, viral infection, bacterial infection, cancer, and drug activities, half-life, or interactions. Exemplary drugs include insulin, biological agents (e.g. Rituximab®) and chemotherapeutics. The methods of the present invention are also useful for evaluation of food or water or other products for contamination with microorganisms or toxins or other contaminants.

In one embodiment, the above described assay methods of this invention are useful in a method for diagnosing sepsis or monitoring the progress thereof. This method is accomplished by performing the desired assay method above with soluble ligands that bind cellular targets including, but not limited to CD64 (N), HLA-DR (Mo), CD11a, CD14 (or CD64)/CD16 (Mo), CD16 (N) and CD 142 (tissue factor) and using soluble ligands and capture medium that bind directly or indirectly the soluble analyte, which may be one or more of IL-6, IL-10, IL-1, TNF-α, neopterin, C-reactive protein, procalcitonin, or activated Protein C.

In another embodiment, the methods above may be adapted for use in diagnosing autoimmune disease or monitoring the progress thereof. According to this aspect of the invention, the assay methods above employ ligands that bind one or more of the cell types including activated T cells and activated B cells by one or more of the cell surface or intracellular antigens that characterize those cells. The methods also use the ligands and capture medium to bind a soluble analyte, which may be one or more of C-reactive protein, an autoantibody, a chemokine, or a cytokine. The selection of chemokine or cytokine used as the soluble reagent may be readily made by one of skill in the art.

In still a further embodiment, the methods of this invention are useful in diagnosing cardiovascular disease or monitoring the progress thereof. Such methods employ as the cellular target one or more of platelet-leucocyte aggregates, or CD 142 (TF) and use ligands that bind thereto. This method is useful in also targeting the soluble analyte, which may be hsC-reactive protein, troponin, or myoglobin. Suitable ligands and capture medium for use in this method may be designed and selected by one of skill in the art given this disclosure.

A method for differential diagnosis of viral and bacterial infections or for use in monitoring the progress thereof employs the assay steps disclosed herein with ligands capable of binding a cellular target, which includes, without limitation, one or more of HLA-DR, CD4/CD8, CD38, CD64(N), or CD14 (or CD64)/CD16 (Mo), CD16 (N). The soluble analyte, which may be one or more of IFNγ, neopterin, or C-reactive protein is detected by ligands and capture medium that bind directly or indirectly these analytes.

In still another embodiment, the methods of the invention are suitable for detecting and monitoring contaminants in fluid, such as water systems, or other liquid products. For example, water may be examined for the presence of bacterial cells by using ligands to cell surface antigens or intracellular antigens of bacterial origin, and for soluble analytes, such as toxins, by using a capture medium on which is associated a legend to the toxin. For example, in one embodiment, the soluble analyte is an enterotoxin, such as cholera, and the cellular target is the enterococcus. One of skill in the art may select other examples of such pollutants and targets for suitable use in methods of this invention.

The methods and compositions of this invention are also adaptable to the diagnosis and monitoring of other diseases and conditions, based on the identification of cellular targets, soluble targets and ligands that bind thereto, as directed by this specification.

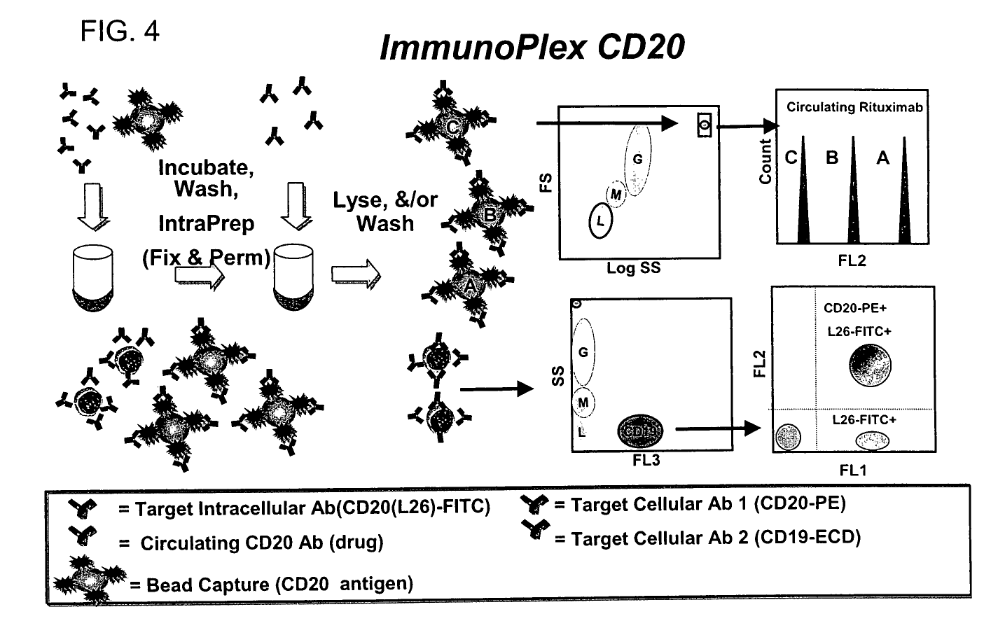

Accordingly, in yet further embodiments illustrated in FIG. 4, the invention provides methods for monitoring treatment of a patient in need thereof being treated with a ligand that binds specifically to the cell surface expressed target CD20. The CD20 monitoring method has various embodiments depending on the breadth of analysis desired. In the simplest format, the method includes obtaining a container containing a sample comprising bodily fluid containing CD20⁺ cells obtained from the patient and adding to the container under conditions and for a time to allow formation of complexes between assay components one or both of the following: 1) permeabilizing cells in the container; and incubating assay components in the container with a first soluble ligand that binds specifically to intracellular CD20 and is conjugated to a first distinguishable fluorescent label under conditions and for a time to allow formation of complexes of intracellular CD20 and the first ligand; or 2) adding to the container a second soluble ligand that binds specifically to B cells and is conjugated to a second fluorescent label under such conditions. The presence of fluorescence from fluorescent labels in complexes formed in the container is then detected to monitor the treatment of the patient. For example, the intensity of fluorescence from only the first label indicates the amount of intracellular CD20 on the B-cells in the sample. In the second instance, the intensity of fluorescence from the second fluorescent label indicates the amount of all B cells in the sample.

Using an imaging analyzer or flow cytometer, the relative intensities of the fluorescence from the fluorescent labels in complexes formed in the container can be substantially simultaneously detected to obtain further information about the patient's response to treatment. For example, when the first alternative is selected, the assay may further include adding to the container one or both of the second soluble ligand and/or a third soluble ligand that binds specifically to CD20⁺ cells. Fluorescence from complexes formed in the container can be analyzed to determine the percentages of B-cells containing CD20 on the surface (FL2) and intracellularly (FL2 and FL1), respectively, in FIG. 4. In yet another example, the method further comprises adding a third ligand that binds specifically to a capture particle in the incubation and detecting the relative intensity of the third fluorescent label to determine the relative amount of circulating treatment ligand present in the patient.

In yet another embodiment, the invention method for monitoring CD20 treatment includes incubating together in a container, such as analysis tube, under conditions and for a time sufficient to allow complex formation between the following assay components: 1) a sample comprising B-cells from blood or bone marrow of the patient; 2) a first ligand that binds specifically to soluble CD20 conjugated to a first distinguishable fluorescent label; 3) a second ligand that binds specifically to B-cells conjugated to a second distinguishable fluorescent label; and 4) a capture particle covalently linked to CD20 antigen. Unbound first and second ligands and unbound components of blood plasma are optionally removed from the container and cells remaining therein are permeabilized. A third ligand that binds specifically to intracellular CD20 conjugated to a third distinguishable fluorescent label is incubated in the container with the complexes formed during the first incubation under conditions and for a time to allow binding between intracellular CD20 and the third ligand. Presence of fluorescence from one or more of the first, second and third fluorescent labels bound to the capture bead and to cells in the sample is substantially simultaneously detected to monitor the treatment of the patient. Ligands that bind specifically to B-cells in the assay methods are selected to bind to a cell surface marker of B-cells in general, such as CD19, CD5, CD22, CD24 and the like.

The sample used in the assay methods may be contained in or obtained from whole blood or bone marrow, in which case, the method may further comprise lysing cells in the container prior to introduction of the third ligand, for example, for binding to intracellular CD20. Relative intensities of the first, second and third fluorescent labels bound to cells and/or to the capture particle can be detected substantially simultaneously by image analyzer or flow cytometry with without separating the complexes formed during the assay prior to the detection.

Due to the nature of the complexes that can form in the container as a result of the invention CD20 monitoring method, valuable information can be learned concerning the success of the patient's treatment. For example, detection of the relative intensity of the first fluorescent label attached to the capture particle can be used to determine the relative amount of circulating treatment ligand present in the blood of the patient. On the other hand, detected intensities of the third and/or second fluorescent labels is directly proportional to the degree of depletion of B-cells in the patient. In addition, the detection of the first fluorescent label is low as compared with detection of the second and third fluorescent labels a blockage of CD20 on B-cells of the patient is indicated.

A more detailed embodiment of the method for monitoring CD20 treatment provides additional information regarding the condition of the patient and includes incubating together in a container under conditions and for a time sufficient to allow complex formation between the following assay components: a sample comprising blood or bone marrow obtained from the patient; a first ligand that binds specifically to CD20+ cells conjugated to a first distinguishable fluorescent label; a second ligand that binds specifically to B-cells conjugated to a second distinguishable fluorescent label; and a capture particle covalently linked to CD20 antigen. After the incubation, cells in the container are permeabilized and assay components are incubated again with a third ligand that binds specifically to intracellular CD20 conjugated to a third distinguishable fluorescent label under conditions and for a time to allow binding between intracellular CD20 and the third ligand. Fluorescence from the first, second or third fluorescent labels in complexes formed in the container is detected to monitor the treatment of the patient, as described above.

In one aspect, the ligands used in the invention methods for monitoring patient treatments can be antibodies, preferably monoclonal antibodies, and the treatment ligand is an antibody approved by the FDA for administration to patients in treatment of a disease associated with expression of CD20. For example, Rituximab® and Bexxar™ monoclonal antibodies are currently approved by the FDA for administration to patients in treatment of B-cell lymphoma and either can be used as the treatment ligand in the invention method to monitor the course of such treatment.

In still another embodiment, the invention provides methods for monitoring treatment of a patient in need thereof being treated with a treatment ligand that binds specifically to the cell surface expressed target CD52. The invention CD52 monitoring method comprises incubating in a container, such as an analysis tube, under conditions and for a time sufficient to allow complex formation between a sample from the patient comprising a bodily fluid containing B-cells, such as from blood or bone marrow, and a treatment ligand that binds specifically to the cell surface expressed target CD52. The invention CD52 monitoring method includes, incubating a sample comprising a bodily fluid containing CD52⁺ cells obtained from the patient in a container under conditions and for a time sufficient to allow complex formation together with a first distinguishable capture particle covalently linked to a CD52 antigen (for binding to circulating drug) and/or a second distinguishable capture particle covalently linked to the treatment ligand (to detect autoantibody formation and any shed CD52 antigen). One of the following assay components is added to the container for the incubation: a first ligand that binds specifically to the expressed target at the binding site of the treatment ligand conjugated to a first distinguishable fluorescent label; a second ligand that binds the expressed target at a different binding site than the treatment ligand conjugated to a second distinguishable ligand; a third ligand that binds specifically to human immunoglobulin conjugated to a third distinguishable fluorescent label. Fluorescence from the fluorescent labels in the complexes formed in the container is detected substantially simultaneously to monitor the treatment of the patient. Optionally, a fourth ligand that binds specifically to B-cells can also introduced for incubation with the sample, and the method further comprises determining the relative intensity of the fourth fluorescent label.

If the sample comprises blood, the method may further comprise removing uncomplexed ligand-fluorescent label conjugates and uncomplexed plasma components from the container prior to the incubation or detection steps of the method. Similarly, if the sample comprises whole blood, red blood cells can be removed from the container or lysed prior to incubation or detection steps of the method. Additionally, if the sample comprises whole blood, the method can further comprise stabilizing the sample prior to b) to prevent artifactual activation, using a procedure known in the art or as illustrated in the Examples herein.

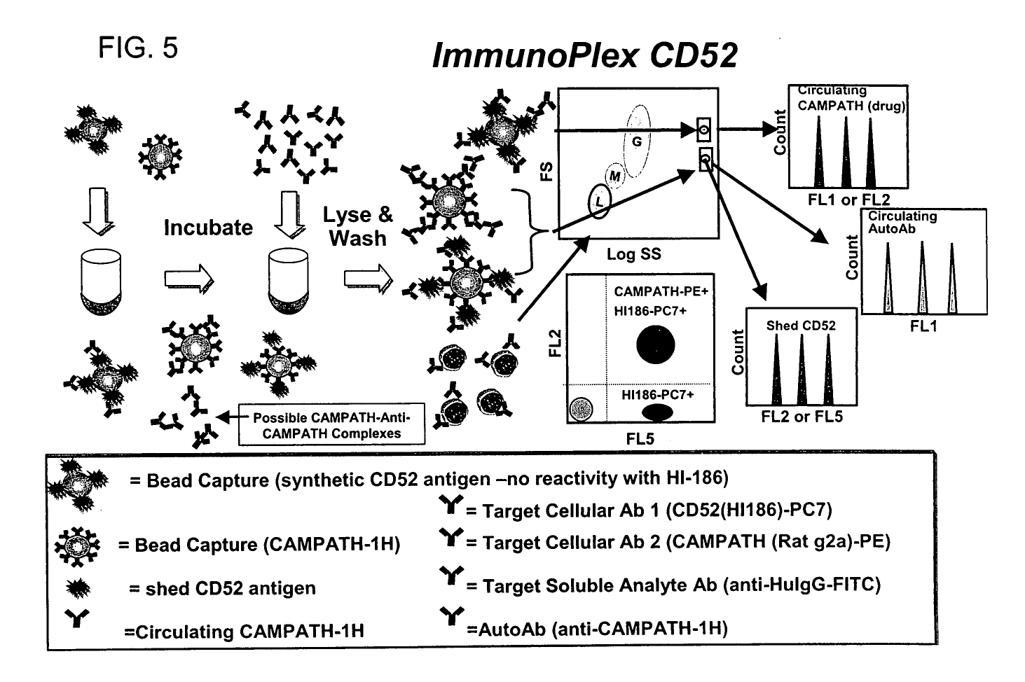

Alternative embodiments of the invention methods for monitoring treatment of a patient being treated with a CD52 ligand can involve addition of further ligands to the incubation and detection steps, as illustrated in FIG. 5. In one example, wherein the first ligand is selected for incubation with the sample in the container, the method can further include adding to the container after the incubation either the first capture particle or a second distinguishable capture particle linked to the treatment ligand, for example, covalently. Alternatively, the third ligand can be added to the container and incubated with either one or both of the first capture particle and the second capture particle. In another example, wherein the second ligand is selected for addition to the container and incubation with the sample, the method further includes adding to the container for incubation with the sample, one or both of the first capture particle and a second distinguishable capture particle linked to the treatment ligand, for example covalently.

In one embodiment, the invention method for monitoring treatment of a patient with a treatment ligand that binds specifically to the cell surface expressed target CD52 can comprise:
a) incubating the following assay components in a container under conditions and for a time sufficient to allow complex formation between:
   1) a sample comprising blood or bone marrow obtained from the patient;
   2) a first distinguishable capture particle linked to a CD52 antigen; and
   3) a second distinguishable capture particle linked to the treatment ligand;
b) incubating in the container the complexes formed in a) under conditions and for a time sufficient to allow binding interaction with the following additional assay components;
   1) a first ligand that binds the expressed target at a different binding site than the treatment ligand, and which is conjugated a first distinguishable fluorescent label;
   2) a second ligand that binds specifically to the expressed target at the binding site of the treatment ligand conjugated to a second distinguishable fluorescent label; and
   3) a third ligand that binds specifically to human immunoglobulin conjugated to a third distinguishable fluorescent label; and
c) detecting substantially simultaneously fluorescence from at least one of the first, second or third fluorescent labels in complexes formed in the container to monitor the treatment of the patient.

As in other embodiments of the invention methods, the detecting can be accomplished using an image processor or flow cytometer to determine the relative intensities of two or more of the fluorescent labels in the container. Due to the nature of the complexes that can form in the container as a result of the invention CD52 monitoring methods, valuable information can be learned concerning the success of the patient's treatment. For example, quantitation of the relative intensities of the third fluorescent label or the second fluorescent label in the sample can be used to determine the relative amount of circulating treatment ligand in the patient. In another example, detection of the relative intensities of the first fluorescent label or the second fluorescent label is useful in determining the relative amount of shed CD52 antigen in the blood of the patient. In still another illustration, detecting the relative intensity of the third fluorescent label can be used to determine the relative amount of circulating autoantibody to the drug or the amount of circulating drug in the patient. The invention methods can also be used to determine the relative amount of CD52 present on the surface of B-cells even when the drug may be masking the CAMPATH epitope.

The measurement of serum levels of treatment ligand (e.g., CAMPATH-1 serum levels) can be used to optimize dose regimens, and also will confirm the evaluation of tumor escape [8,9]. The potential for anti-idiotype antibodies, though less problematic with humanized monoclonal antibodies such as CAMPATH-1H, may also be monitored. In addition, due to the toxicity of this treatment (e.g., extensive depletion of lymphocytes), the ability to quantitate differences in the level of CD52 expression may allow stratification of responders to non-responders.

In one aspect, in the invention methods for monitoring anti-CD52 treatment, the ligands can be antibodies, preferably monoclonal antibodies and the treatment ligand is one approved by the FDA for administration to patients in treatment of a disease associated with expression of CD52. For example, CAMPATH-1H monoclonal antibody is currently approved by the FDA for administration to patients in treatment of B-cell chronic lymphocytic leukemia and can be used as the treatment ligand in the invention method to monitor the course of such treatment. In such a case a monoclonal antibody that does not bind to the CAMPATH-1 epitope of CD52, such as CD52 antibody clone HI186, can conveniently be used as the second ligand in the assay, although any ligand that binds to an epitope to which CAMPATH-1H does not bind can also be used for this purpose. The synthetic antigen attached to the first capture bead may also be selected to have no reactivity with the epitope of CD52 to which the second ligand binds.

In still another embodiment the invention provides methods for monitoring side effects of heparin therapy in a patient, such as heparin-induced thrombocytopenia (HIT). The invention method includes incubating in a container under conditions and for a time sufficient to allow complex formation between the following assay components: 1) a sample comprising stabilized whole blood of the patient; 2) a first distinguishable capture particle linked to a heparin:platelet factor 4 (H:PF4) complex; 3) a first ligand that binds specifically to a platelet activation antigen conjugated to a first fluorescent label, and 4) a second ligand that binds specifically to all platelets and is conjugated to a second fluorescent label. After the incubation, unbound first and second ligand and unbound plasma components are optionally removed from the container. Then a third ligand that binds specifically to human immunoglobulin conjugated to a third fluorescent label is added to contents of the container and the contents are incubated under conditions and for a time sufficient to allow complex formation therebetween. The presence of the first fluorescent label, second fluorescent label and third fluorescent label in complexes formed in the container is detected to monitor heparin therapy of the patient. The detection can include using an image analyzer or flow cytometer to detect the percentage of first and second fluorescent labels in the complexes to determine the percent of platelet activation antigen in the blood of the patient. Alternatively, the detecting can involve detecting the mean fluorescence intensity of the third fluorescent label bound to the capture particle to assess the amount of anti-heparin autoantibody in the blood of the patient. Alternatively, the detecting step can include detecting the ratio of red cells to platelets in the sample to determine platelet concentration in the sample.

A useful monoclonal antibody for use as the first ligand is an anti-CD62p antibody. CD62p antibody is used to target platelet activation, and -CD41 conjugated to a second distinguishable fluorescent label can be used as a gating reagent for platelets.

These methods, which allow for substantially simultaneous analysis of relevant cellular and soluble targets, cellular antigens, cell characteristics and hematology parameters, provide a more complete picture than do prior art methods of a patient's medical status with regard to both cellular and soluble mediators, activators or inhibitors. This invention permits multiple assays to be conducted in a single test tube or microtiter plate and allows a comprehensive snapshot of patient status or drug effects. The advantages of the methods of this invention include decreased sample size (e.g., blood) requirements, which are particularly important for pediatric and geriatric patients, increased accuracy or clinical monitoring, increased throughput efficiency, reduced time and labor to conduct the tests, and decreased overall cost to a patient. For example, the ability to assess activated immune cells in combination with a variety of soluble analytes can improve both the diagnosis and monitoring of the above-noted diseases.

If the sample comprises blood, the method may further comprise removing uncomplexed ligand-fluorescent label conjugates and uncomplexed plasma components from the container prior to the incubation or detection steps of the method. Additionally, the method can further comprise stabilizing the sample prior to b) to prevent artifactual activation, using a procedure known in the art or as illustrated in the Examples herein.

Kits

For convenience, the conventional reagents for high throughput assays or other diagnostic assays useful according to this invention may be provided in the form of kits. In yet another aspect of this invention, a kit is provided for performance of the above-described methods. Preferably such kits are employed for performing the diagnostic methods of this invention and/or monitoring therapy. However, such kits can be assembled for research purposes also. Thus, a kit of the present invention desirably contains the components taught above, e.g., at least one soluble ligand that binds a cellular target in the sample; at least one soluble ligand that binds a soluble analyte in the sample or at least one competing soluble analyte (preferably labeled); and a solid phase capture medium that binds directly to the soluble analyte, indirectly to the soluble analyte, or to the soluble ligand that binds to the soluble analyte. The kits also include instructions for performing the particular assay, various diluents and buffers, and signal-generating reagents, such as fluorescent labels, enzyme substrates, cofactors and chromogens. Other components may include indicator charts for calorimetric comparisons, disposable gloves, decontamination instructions, applicator sticks or containers, and a sample preparatory cup.

In one embodiment of the present invention, a kit useful for the performance of the above-described sandwich assay includes, as a component, a solid phase capture medium associated with multiple first ligands that bind the soluble analyte. Another kit component is the soluble ligand that binds the cellular target and is associated with a first detectable label. The kit further comprises a third ligand that is capable of binding to the soluble analyte-first ligand-capture medium complex. The third ligand associated with a second detectable label.

In another embodiment, a kit for performing one of the competitive inhibition assays described above, contains a first ligand associated with a first label. Multiple of the first ligands are capable of binding to a single cellular target. Another component is a second ligand associated with a second label. The second ligand is capable of binding a soluble analyte. Still another component is the solid phase capture medium associated with multiple of the soluble analytes immobilized thereon.

In another embodiment, a kit for performing another of the competitive inhibition assays described above, contains a first ligand associated with a first label. Multiple of the first ligands are capable of binding to a single cellular target. Another component is a competing analyte associated with a second label. Still another component is the solid phase capture medium on which are immobilized multiple of ligands capable of binding to the soluble analyte (either competing soluble analyte or soluble analyte naturally occurring in the sample).

In yet another embodiment, a kit for performing the immune complex assay of this invention includes a first ligand capable of binding to a first cellular target and providing a first detectable signal; a second ligand capable of binding to the soluble analyte and providing a second detectable signal; a third ligand capable of binding to the same soluble analyte; a solid phase capture medium on which is immobilized multiple fourth ligands, the fourth ligands capable of binding to the third ligands.

Such kits are useful for evaluating blood samples for purposes of determining disease states associated with inappropriate types or numbers of blood cells, blood cell types or bound components or soluble antigens or analytes thereof. Thus, such a kit will be useful in conducting the diagnostic assays discussed herein, e.g., in determining the status of treatment of an illness characterized by inappropriate cell target or soluble analyte expression in a blood sample. Such a diagnostic kit contains the dyes, ligands, capture medium, and other components of the methods of this invention. Such kits also contain labels, exemplified above, pre-attached to the other components of the specific assay to be performed, or provided separately for attachment to a selected component, e.g., a substrate. Alternatively, such kits may contain a simple mixture of such compositions or means for preparing a simple mixture.

Such kits provide a convenient, efficient way for a clinical laboratory to screen blood samples or other biological samples containing cells according to this invention.

One of skill in the art may be expected to vary the components of these diagnostic kits in obvious ways based on the knowledge in the art coupled with this disclosure. Such varied components are included in this embodiment of the invention.

The kit further comprising at least one of the following additional components selected from the group consisting of suitable vessels for containing samples, suitable controls or tables of normal or disease-characteristic values of activated platelets; an anti-coagulant or coagulation pathway inhibitor, other reagents suitable for the performance of flow cytometric analyses and combinations thereof; suitable diluents and buffers for the samples, disposable gloves, decontamination instructions, applicator sticks or containers, and sample preparatory cups.

### EXAMPLES

These examples demonstrate the use of the methods and compositions of the invention and the analysis thereof. The data reported in these Examples demonstrates that the novel methods of this invention have performance parameters that permit improved efficient and substantially simultaneous analysis of samples with multiple types of targets. These examples are illustrative and do not limit the scope thereon. One of skill in the art will appreciate that although specific reagents and conditions are outlined in the following examples, modifications as described above can be made to provide the compositions of this invention or processes for use thereof.

### Example 1

ImmunoPlex Multiple Analyte Sandwich Immunoassay.

The following example was performed to test combined use of flow cytometry and immunoassay technology for substantially simultaneous assay of cellular marker expression profile (immunophenotyping), quantitation of soluble (serum markers), and white blood cell percentage in a single assay tube.

To a sample (100 uL) of EDTA-treated whole blood are added the following reagents:
(a) The capture medium is 50 μl of a six-bead polystyrene microsphere bead population with distinct fluorescence intensities. The microspheres or beads are generally larger than 3.6 μm and smaller than 10 μm. Antibodies are individually covalently attached to a subset of the beads by conventional methods to create the antibody-conjugated fluorescence capture beads with bead specificity for IL-2, IL-4, IL-5, IL-10, TNF-α or IFNγ.
(b) Phycoerythrin (PE)-conjugated anti-human detector soluble ligand (50 ul/test) with an antibody specificity for anti-IL-2, anti-IL-4, anti-IL-5, anti-IL-10, anti-TNF-α, or anti-IFN-γ.
(c) A soluble ligand to the cellular target, i.e., 20 μl of anti-CD45-FITC (Beckman Coulter).

This reagent mixture is incubated for between about 1 hour to 3 hours at room temperature with gentle mixing and also is protected from light. For comparison, a parallel sample was created by substituting plasma for whole blood.

Once incubated, the red blood cells in the sample were lysed by the addition of ImmunoPrep™ reagents (Beckman Coulter). The samples were then subjected to cytometric flow analysis using a Beckman Coulter FC 500 Flow Cytometer without any further manipulation or separation of the various complexes formed among the reagents in the sample and the data was collected.

The results showed no significant difference in cytokine values when capture bead/detector reagents were incubated in plasma or in whole blood, with or without fluorochrome anti-CD markers. The assay range for the cytokines evaluated was from 0 to 5000 pg/mL. There was also no effect of the beads on cellular scatter parameters or on the determination of CD expression, as measured by mean fluorescence intensity. Thus the data provided reliable information on white blood cell percentages, cell surface expression of marker proteins, and serum cytokine levels in a single tube analysis format.

### Example 2

ImmunoPlex Single Analyte Capture Immunoassay

A sample of whole blood (100 μl) collected in the anticoagulant EDTA, is kept on ice or at room temperature throughout this experiment. Parallel samples were created by substituting plasma or buffer for the whole blood. To these samples were added the following reagents:
(a) 10 μl of capture beads. As capture bead, a single non-fluorescent, paramagnetic, polystyrene microsphere is used to create six populations with bound antibodies to the soluble analyte IL2, i.e., human IL-2 antibodies. The beads are larger than 3.61 μm and smaller than 10 μm.
(b) 20 μl of a ligand to the cellular target CD14, which is an antibody labeled with fluorescent isothiocyanate (anti-CD14-FITC) (Beckman Coulter); and
(c) 10 μl of a ligand to the cellular target CD45, which is an antibody labeled with phycocyanin-5 (anti-CD45-PC5) (Beckman Coulter).

The samples are then incubated for 60 minutes mixing twice every 30 seconds (or alternatively rocking).

Thereafter 10 μl of a soluble ligand to the soluble analyte IL-2, i.e., phycoerythrin (PE) conjugated anti-IL2 reporter antibody, was added to the samples of buffer, plasma and whole blood and the samples were incubated for 30 minutes, again mixing twice per 30 seconds (or rocking).

The whole blood samples were then lysed using ImmunoPrep™ reagents (Beckman Coulter) and all samples were subjected to cytometric flow analysis on a Cytomics FC500 or Coulter® XL/MCL™ flow cytometer without any further manipulation or separation of the various complexes formed among the reagents in the sample and the data was collected.

To assess bead effect on light scatter and CD expression, the samples were processed with and without beads to assess any impact on cellular light scatter patterns and mean fluorescent intensity (MFI). Lymphocytes, monocytes and granulocytes were examined, as being considered worst case scenario due to potential phagocytosis of particles.

The data collected showed there was no difference in values with respect to light scatter and MFI obtained in plasma compared to whole blood, confirming that phagocytosis of beads was inhibited.

The assay was performed with and without anti-IL-2 beads, with and without soluble target antibody (IL2-PE), and with and without cellular target antibody (CD14-FITC). Isotypic controls were used as negative controls. As expected, a distinctly different regression equation was seen from plotting the results obtained when the sample media was buffer as compared to plasma or whole blood, indicating “matrix effects”. The addition of beads and soluble target antibody did not adversely impact cellular scatter pattern or antigenic expression.

### Example 3

ImmunoPlex CD20

This example and FIG. 4 illustrate use of the invention methods to monitor treatment of a patient with a ligand (antibody) that binds to CD20. Whole blood, fine needle aspirates or bone marrow is placed in an analysis tube and components are stained by coincubation with the following components:
a) an antibody that binds specifically to the cell surface expressed target, CD20, and which has been conjugated to a first distinguishable fluorescent label, fluorochrome 1 (Ab-FL1) (clone HRC20)-PE);
b) a second antibody that identifies the cell lineage (i.e. CD19) and which is conjugated to a second distinguishable fluorescent label, fluorochrome 2 (Ab-FL2)(CD19-ECD®) a direct antibody conjugate; and
c) a capture bead (as described in Example 2 above) that can be discriminated from cells by physical or fluorescent characteristics and is linked to CD20 antigen, for example, covalently (and/or a preserved cell that has a known expression of CD20 and which can be discriminated from all other cells in the mixture by physical and/or fluorescent characteristics). The mixture is incubated for a sufficient time to allow optimal binding of components in the analysis tube. Excess (unbound) components a) and b) and non-bound plasma components are removed through washing.

The sample is then treated with a fixation and permeabilization reagent (e.g. IntraPrep™ reagent, Beckman Coulter) to allow entry into cells for tagging of intracellular marker proteins, and an antibody that binds specifically to intracellular CD20 and which has been conjugated to a third distinguishable fluorescent label, fluorochrome 3 (Ab-FL3) (clone L26)-FITC) is introduced into the analysis tube (Mason, D. Y. et al. 1990. Am J Pathol 136:1215-1222). Incubation proceeds to allow optimal binding of Ab-FL3. The sample may be lysed and/or washed and is then analyzed on a flow cytometer or imaging system to enable the comparison and relative quantitation of cell surface expression, intracellular expression, as well as circulating CD20 expression by comparison of the relative fluorescent staining of a combination of one or more bead and/or preserved cell combinations using the procedure illustrated schematically in FIG. 4. Utilization of calibrator beads or cells can further enable a quantitative measurement to be made. Additionally hematology differential parameters can be ascertained.

### Example 4

This example and FIG. 5 illustrate the invention assay procedure as used to monitor the course of treatment of a patient using CD52 antibody, CAMPATH-1. Whole blood, bone marrow or single cell suspensions are first placed in a single container and incubated with a first capture particle, such as a distinguishable capture bead, that has been covalently linked to a synthetic CD52 antigen (1Bead-Ag) that contains the epitope to which CAMPATH-1 binds, and with a second capture particle that is covalently linked to CAMPATH-1, the treatment antibody (2Bead-Drug). The order of introduction of the two is immaterial. The mixture is incubated (first incubation period) for a sufficient time to allow optimal binding of the capture beads with their targets in the sample. Bead-Ag will bind with any circulating drug, Bead-Drug will bind with any circulating anti-CAMPATH-1 antibodies or shed receptor in the sample.

For analysis, the sample is then stained in the analysis tube with a series of antibodies labeled with distinguishable fluorochromes:
a) A treatment antibody that binds specifically to the cell surface expressed target, e.g. CD52 conjugated to first fluorescent label FL2 (CAMPATH-1-PE)(Ab-FL2);
   b) a second antibody that binds the same expressed target antigen (CD52) at a different site than CAMPATH-1 and which is conjugated to a second fluorescent label (HI186)—PC7) (Ab-FL5);
c) To detect any target soluble analyte, a third antibody that binds specifically to human immunoglobulin (e.g. anti-HuIgG-FITC), and which is conjugated to a third fluorescent label (Ab-FL1); and
d) A fourth marker for cell lineage may also be added at this time (Ab-FL4; e.g. CD19-ECD (not shown in FIG. 5).

The sample is incubated in the analysis tube (i.e. second incubation period) for a sufficient time to allow optimal binding between components. The sample is then lysed and/or washed to remove excess Ab-FLs, non-bound plasma components and red cells and the contents of the container are analyzed on a flow cytometer. The amounts of CD52 present on the cell surface and circulating in the subject's blood can be assessed along with the appearance of any anti-CAMPATH-1 response. Utilization of calibrator beads or cells further enables a quantitative measurement to be made. The entire analysis is performed on the same instrument and without separating the complexes formed in the sample tube. Additionally hematology differential parameters can be ascertained.

### Example 5

This example illustrates use of the invention methods to monitor treatment of a patient with heparin. Whole blood is first stabilized (ThombFix, or CTAD (citrate, theophylline, adenosine, and dipyridamole cocktail) (Becton Dickenson, CA) to prevent any artifactual activation. A bead is covalently linked to heparin:platelet factor 4 (H:PF4) complexes and/or to a preserved cell with H:PF4 and which can be discriminated from all other cells in the mixture by physical and/or fluorescent characteristics (Bead-H:PF4). A fluorochrome-conjugated antibody (Ab-FL1) that binds specifically to a platelet activation antigen (e.g. CD62p-FITC) is prepared. The beads and fluorochrome-conjugated antibody are added to the stabilized blood in an analysis tube. A second fluorochrome-conjugated antibody that binds specifically to an additional platelet marker (e.g. CD41-PC7) may also be added to the analysis tube at this time (Ab-FL4;). The sample is incubated for a sufficient time to allow optimal binding of Bead-H:PF4 with any anti-H:PF4 autoantibodies. A subsequent wash removes any excess Ab-FL1, Ab-FL2 or non-bound plasma components. To detect the anti-H:PF4 autoantibodies bound by the Bead-H:PF4, a third antibody that binds specifically to human immunoglobulins conjugated to a third fluorescent label FL2 (anti-HuIg-PE) is added to the analysis tube and the mixture is incubated to allow binding to the full extent to occur.

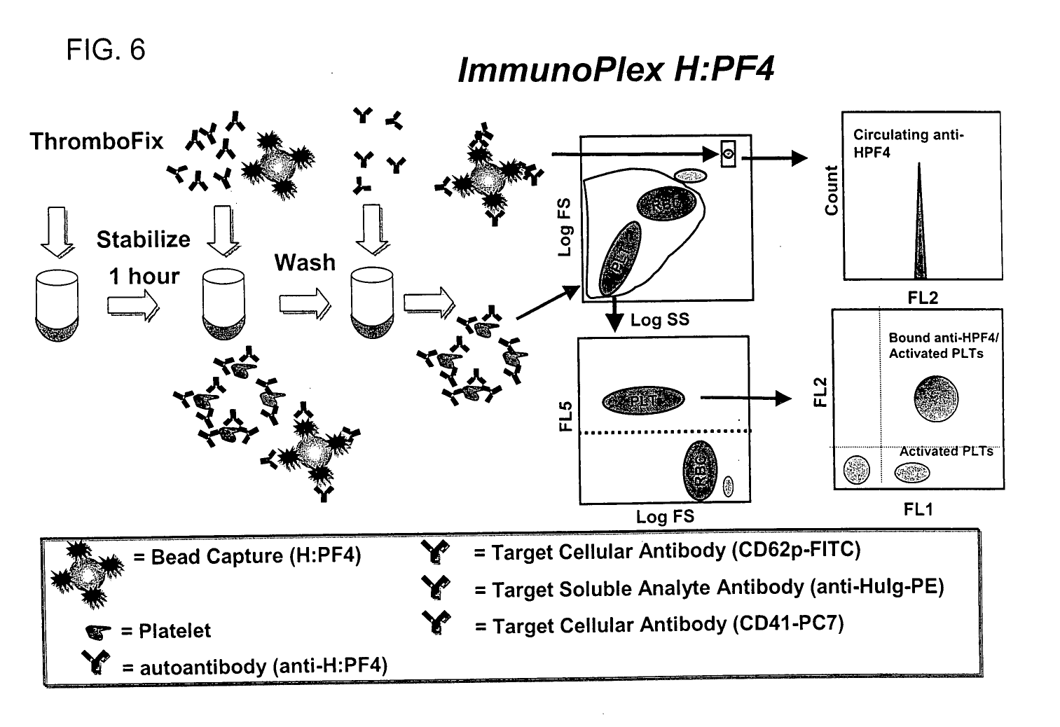

The sample is then analyzed on a flow cytometer using the procedure illustrated schematically in FIG. 6. Detection of platelet activation is assessed by determining the percent of CD62p, autoantibody measurement is quantified by the mean fluorescent intensity of the complex including Bead-H:PF4:anti-H:PF4:anti-HuIg-PE (FL2). Platelet concentration is determined by the ratio of the red cells to the platelets as per the guidelines of the International Society for Laboratory Hematology (ISLH) (International Council for Standardization in Haematology Expert Panel on Cytometry and International Society of Laboratory Hematology Task Force on Platelet Counting. Platelet counting by the RBC/platelet ratio method: a reference method. Am J Clin Pathol. 2001; 115:460-464) Without sample separation prior to analysis, platelet count, platelet activation and presence of anti-H:PF4 antibodies are analyzed in a single tube for the determination of HIT. (PLT=platelets; RBC=red blood cells).

All documents cited above are incorporated by reference herein. The compositions and processes of the present invention are encompassed by the scope of the claims appended hereto.

### REFERENCE LIST

1. Giles, F. J., J. M. Vose, K. A. Do, M. M. Johnson, T. Manshouri, G. Bociek, P. J. Bierman, S. M. Brien, M. J. Keating, H. M. Kantarjian, J. O. Armitage, and M. Albitar. 2003. Circulating CD20 and CD52 in patients with non-Hodgkin's lymphoma or Hodgkin's disease. Br J Haematol 123:850-857.
2. Clarke, L. E., M. G. Bayerl, W. C. Ehmann, and K. F. Helm. 2003. Cutaneous B-cell lymphoma with loss of CD20 immunoreactivity after rituximab therapy. J Cutan Pathol 30:459-462.
3. Kennedy, G. A., S. K. Tey, R. Cobcroft, P. Marlton, G. Cull, K. Grimmett, D. Thomson, and D. Gill. 2002. Incidence and nature of CD20-negative relapses following rituximab therapy in aggressive B-cell non-Hodgkin's lymphoma: a retrospective review. Br J Haematol 119:412-416.
4. Davis, T. A., D. K. Czerwinski, and R. Levy. 1999. Therapy of B-cell lymphoma with anti-CD20 antibodies can result in the loss of CD20 antigen expression. Clin Cancer Res 5:611-615.
5. Mason, D. Y., B. Comans, J. L. Cordell, M. A. Verhoeven, and D. van. 1990. Antibody L26 recognizes an intracellular epitope on the B-cell-associated CD20 antigen. Am J Pathol 136:1215-1222.
6. Dumont, F. J. 2002. CAMPATH (alemtuzumab) for the treatment of chronic lymphocytic leukemia and beyond. Expert Rev Anticancer Ther 2:23-35.
7. Taylor, V. C., M. Sims, S. Brett, and M. C. Field. 1997. Antibody selection against CD52 produces a paroxysmal nocturnal haemoglobinuria phenotype in human lymphocytes by a novel mechanism. Biochem J 322 (Pt 3):919-925.
8. Birhiray, R. E., G. Shaw, S. Guldan, D. Rudolf, D. Delmastro, P. Santabarbara, and L. Brettman. 2002. Phenotypic transformation of CD52(pos) to CD52(neg) leukemic T cells as a mechanism for resistance to CAMPATH-1H. Leukemia 16:861-864.
9. Rebello, P. and G. Hale. 2002. Pharmacokinetics of CAMPATH-1H: assay development and validation. J Immunol Methods 260:285-302.
10. Rebello, P. R., G. Hale, P. J. Friend, S. P. Cobbold, and H. Waldmann. 1999. Anti-globulin responses to rat and humanized CAMPATH-1 monoclonal antibody used to treat transplant rejection. Transplantation 68:1417-1420.
11. Ginaldi, L., M. De, E. Matutes, N. Farahat, R. Morilla, M. J. Dyer, and D. Catovsky. 1998-Levels of expression of CD52 in normal and leukemic B and T cells: correlation with in vivo therapeutic responses to Campath-1H. Leuk Res 22:185-191.
12. Reilly, R. F. 2003. The pathophysiology of immune-mediated heparin-induced thrombocytopenia. Semin Dial 16:54-60.
13. DeBois, W. J., J. Liu, L. Y. Lee, L. N. Girardi, C. Mack, A. Tortolani, K. H. Krieger, and O. W. Isom. 2003. Diagnosis and treatment of heparin-induced thrombocytopenia. Perfusion 18:47-53.
14. Chong, B. H. 2003. Heparin-induced thrombocytopenia. J Thromb Haemost 1: 1471-1478.
15. Alberio, L., S. Kimmerle, A. Baumann, B. M. Taleghani, F. D. Biasiutti, and B. Lammle. 2003. Rapid determination of anti-heparin/platelet factor 4 antibody titers in the diagnosis of heparin-induced thrombocytopenia. Am J Med 114:528-536.
16. Chilver, S., B. Lammle, and L. Alberio. 2004. Titre of anti-heparin/PF4-antibodies and extent of in vivo activation of the coagulation and fibrinolytic systems. Thromb Haemost 91:276-282.
17. Gobbi, G., P. Mirandola, P. L. Tazzari, E. Talarico, L. Caimi, G. Martini, S. Papa, R. Conte, F. A. Manzoli, and M. Vitale. 2004. New laboratory test in flow cytometry for the combined analysis of serologic and cellular parameters in the diagnosis of heparin-induced thrombocytopenia. Cytometry 58B:32-38.
18. Tazzari, P. L., F. Ricci, M. Vitale, F. Malferrari, A. Salama, P. Schwind, and R. Conte. 2002. Heparin-induced thrombocytopenia: detection of antiheparin/PF4 antibodies by means of heparin/PF4-coated beads and flow cytometry. Transfus Med 12:193-198.
19. Lindhoff, L., F. Gerdsen, H. Ackermann, and R. Bauersachs. 2001. Determination of heparin-platelet factor 4-IgG antibodies improves diagnosis of heparin-induced thrombocytopenia. Br J Haematol 113:886-890.
20. Vun, C. M., S. Evans, and C. N. Chesterman. 2001. Anti-PF4-heparin immunoglobulin G is the major class of heparin-induced thrombocytopenia antibody: findings of an enzyme-linked immunofiltration assay using membrane-bound hPF4-heparin. Br J Haematol 112:69-75.
21. Amiral, J., C. Pouplard, A. M. Vissac, J. M. Walenga, W. Jeske, and Y. Gruel. 2000. Affinity purification of heparin-dependent antibodies to platelet factor 4 developed in heparin-induced thrombocytopenia: biological characteristics and effects on platelet activation. Br J Haematol 109:336-341.
22. Untch, B., S. Ahmad, W. P. Jeske, H. L. Messmore, D. A. Hoppensteadt, J. M. Walenga, H. Lietz, and J. Fareed. 2002. Prevalence, isotype, and functionality of antiheparin-platelet factor 4 antibodies in patients treated with heparin and clinically suspected for heparin-induced thrombocytopenia. The pathogenic role of IgG. Thromb Res 105:117-123.
23. Vitale, M., P. Tazzari, F. Ricci, M. A. Mazza, G. Zauli, G. Martini, L. Caimi, F. A. Manzoli, and R. Conte. 2001. Comparison between different laboratory tests for the detection and prevention of heparin-induced thrombocytopenia. Cytometry 46:290-295.
24. Rodriguez-Caballero, A., Garcia-Montero, A., Bueno, C. and A. Orfao.

2003. Flow cytometric analysis of cytokine responses in stimulated whole blood: simultaneous quantitation of TNF-α-secreting cells and soluble cytokines. Current Protocols in Cytometry 9.21.1-21.
25. Kawate, Yasunori and Hiraoka-cho, Kakogawa (Sysmex European Patent; Publication NO. EP 01/387171-A1). Analyzers and methods of analyzing blood.
26. Hechinger, Mark (Patent Application Number—243515/10; Filed 2002-09-13; Publication number—20030194818/US-A1).
27. International Council for Standardization in Haematology Expert Panel on Cytometry and International Society of Laboratory Hematology Task Force on Platelet Counting. Platelet counting by the RBC/platelet ratio method: a reference method. Am J Clin Pathol. 2001; 115:460-464.

1-62. (canceled)

63. A method for evaluation of a biological sample containing at least one cell type bearing at least one cellular target and at least one soluble analyte, the steps comprising:
(a) adding to a single container said sample with
(i) at least one soluble ligand that binds to said cellular target,
(ii) at least one soluble ligand that binds to said soluble analyte, or at last one competing soluble analyte; and
(iii) a solid phase capture medium that binds directly to said soluble analyte, indirectly to said soluble analyte, or to said soluble ligand that binds to said soluble analyte; and
(b) simultaneously analyzing said sample (a) without physically separating the complexes comprising a complex that forms between said cellular target and said at least one soluble ligand, and a complex that forms between said capture medium bound directly to said soluble analyte, or between said capture medium bound indirectly to said soluble analyte, or between said capture medium bound to said soluble ligand that is bound to said soluble analyte.

64. The method of claim 63, further comprising in (a):
(i) adding to said sample a solid phase capture medium on which are immobilized multiple first ligands that are capable of binding to said soluble analyte, and wherein said capture medium, said first ligand, and said soluble analyte form a first complex;
(ii) adding to sample obtained from (i) a soluble second ligand that binds specifically to said cellular target, wherein each second ligand is associated with a first detectable label, and wherein multiple second ligands can bind to a single target cell to form a second complex;
(iii) adding to sample obtained from (ii) a third ligand associated with a second detectable label and that binds specifically to said first complex, wherein said third ligand and said first complex form a third complex, and
(b) simultaneously analyzing sample obtained from (aiii) without physically separating said complexes by discriminating between said third complex and said second complex.

65. The method of claim 63, wherein said soluble analyte is selected from the group consisting of a serum marker, a protein, a virus, a hormone, a lipid, a nucleic acid sequence, a carbohydrate, a pharmaceutical drug, a toxin, and an antigen shed from a cell.

66. The method of claim 63, wherein said method is useful in diagnosis of a disease or condition selected from the group consisting of sepsis, inflammation, autoimmune disease, cardiovascular disease, viral infection, bacterial infection, and drug interaction.

67. A method for diagnosing sepsis or monitoring the progress thereof by performing the method of claim 63, wherein said cellular target is selected from the group consisting of CD64 (N), HLA-DR (Mo), CD11a, CD14/CD16, CD142; and said soluble target is selected from the group consisting of IL-6, IL-10, IL-1, TNF-alpha, neopterin, C-reactive protein, procalcitonin, and activated Protein C.

68. A method for diagnosing autoimmune disease or monitoring the progress thereof by performing the method of claim 63, wherein said cellular target is selected from the group consisting of activated T cells and activated B cells; and said soluble target is selected from the group consisting of C-reactive protein, a chemokine, and a cytokine.

69. A method for diagnosing cardiovascular disease or monitoring the progress thereof by performing the method of claim 63, wherein said cellular target is selected from the group consisting of platelet-leucocyte aggregates, CD142 (TF); and said soluble target is selected from the group consisting of hsC-reactive protein, troponin, and myoglobin.

70. A method for differential diagnosis of viral and bacterial infections or monitoring the progress thereof by performing the method of claim 63, wherein said cellular target is selected from the group consisting of HLA-DR, CD4/CD8, CD64 (N), CD14/CD16; and said soluble target is selected from the group consisting of IFN-gamma, neopterin, and C-reactive protein.

71. A method for monitoring treatment of a patient in need thereof with a treatment ligand that binds specifically to the cell surface expressed target CD20, comprising:
a) obtaining a container containing a sample comprising bodily fluid containing CD20⁺ cells obtained from the patient;
b) performing one of the following:
i) incubating assay components in the container with a first soluble ligand that binds specifically to CD20 and is conjugated to a first distinguishable fluorescent label under conditions and for a time to allow formation of complexes of CD20 and the first ligand; or
ii) adding to the container a second soluble ligand that binds specifically to B cells and is conjugated to a second fluorescent label under conditions and for a time to allow formation of complexes between assay components; or
iii) the following combination of steps:
adding to the container a capture particle linked to CD20 antigen;
permeabilizing cells in the container; and
incubating assay components in the container with a third soluble ligand that binds specifically to intracellular CD20 and is conjugated to a third distinguishable fluorescent label under conditions and for a time to allow formation of complexes of intracellular CD20 and the first third ligand; and
c) detecting the presence of fluorescence from fluorescent labels in complexes formed in the container to monitor the treatment of the patient.

72. The method of claim 71, wherein performance of b) iii) is selected, further comprising, prior to performance of b) iii), adding to the container:
a) the second soluble ligand that binds specifically to B-cells conjugated to the second distinguishable fluorescent label; and/or a third soluble ligand that binds specifically to CD20⁺ cells, and/or the capture particle that contains the CD20 antigen; and
b) incubating contents of the container under conditions and for a time sufficient to allow complex formation between assay components therein.

73. The method of claim 71, wherein the sample is whole blood.

74. The method of claim 71, wherein the ligands are antibodies.

75. The method of claim 71, wherein the treatment ligand is Rituximab® or Bexxar™ monoclonal antibody.

76. The method of claim 71, wherein the relative intensities of the fluorescent labels in the complexes are detected by a fluorometer or flow cytometer.

77. A method for monitoring treatment of a patient in need thereof with a treatment ligand that binds specifically to the cell surface expressed target CD52, comprising:
a) obtaining a container containing a sample comprising a bodily fluid containing CD52⁺ cells obtained from the patient;
b) incubating the sample in the container under conditions and for a time sufficient to allow complex formation with:
i) a first ligand that binds specifically to the expressed target at the binding site of the treatment ligand conjugated to a first distinguishable fluorescent label and
ii) one assay component selected from:
a) a second ligand that binds the expressed target at a different binding site than the treatment ligand conjugated to a second distinguishable ligand;
b) a third ligand that binds specifically to human immunoglobulin conjugated to a third distinguishable fluorescent label; and
c) a first distinguishable capture particle linked to a CD52 antigen; and
d) a second distinguishable capture particle linked to the treatment ligand; and
c) detecting substantially simultaneously fluorescence from the fluorescent labels in the complexes formed in the container to monitor the treatment of the patient.

78. The method of claim 77, wherein in b)ii) the second ligand is selected, said method further comprising:
in a) adding to the container either the first capture particle or a second distinguishable capture particle linked to the treatment ligand; or
in b ii) adding the third ligand and either one or both of the first capture particle and the second capture particle.

79. The method of claim 77, wherein in b)ii) the second ligand is selected, the method further comprising in a), adding to the container one or both of the first capture particle and a second distinguishable capture particle linked to the treatment ligand.

80. The method of claim 77, wherein in b)ii) the third ligand is selected, the method further comprising in a), adding to the container one or both of a first distinguishable capture particle linked to a CD52 antigen and the second capture particle.

81. The method of claim 77, wherein the treatment ligand is CAMPATH-1H monoclonal antibody and the first ligand is an antibody that binds specifically to an epitope of CD52 that is different from the epitope bound by CAMPATH-1.

82. The method of claim 77, wherein the treatment is treatment of B-cell chronic lymphocytic leukemia.

83. A method for monitoring side effects of heparin therapy in a patient in need thereof, said method comprising:
a) incubating the following assay components in a container under conditions and for a time sufficient to allow complex formation between:
i) a sample comprising whole blood of the patient;
ii) a distinguishable capture particle linked to heparin:platelet factor 4 complex;
iii) a first soluble ligand that binds specifically to a platelet activation antigen and is conjugated to a first fluorescent label; and
iv) a second soluble ligand that binds specifically to platelets and is conjugated to a second fluorescent label;
b) incubating in the container under conditions and for a time sufficient to allow complex formation between the complexes formed in a) and a third ligand that binds specifically to human immunoglobulins and is conjugated to a third distinguishable fluorescent label, thereby forming a mixture of complexes therein; and
c) detecting fluorescence from the first fluorescent label, second fluorescent label or the third fluorescent label in the mixture of complexes to monitor heparin therapy in the patient.

84. The method of claim 83, wherein the sample comprises whole blood or platelet-rich plasma.

85. The method of claim 83, wherein the ligands are antibodies.

86. The method of claim 83, wherein the detecting is by fluorometer or flow cytometer.

87. A kit comprising:
a) at least one soluble ligand that binds a cellular target in a liquid sample;
b) at least one soluble ligand that binds a soluble analyte in said sample or at least one soluble analyte associated with a detectable label; and
c) a solid phase capture medium that binds directly to said soluble analyte, indirectly to said soluble analyte, or to said soluble ligand that binds to said soluble analyte.

88. The kit of claim 87, further comprising a container comprising one or a mixture of components a) through c).

89. The kit of claim 87, wherein the kit is for monitoring treatment of a patient with a treatment ligand that binds specifically to the cell surface expressed target CD20, and comprises:
a) a first soluble ligand that binds specifically to CD20; and one or more of the following:
b) a second soluble ligand conjugated that binds specifically to intracellular CD20+ cells;
c) a third soluble ligand that binds specifically to B cells; and
d) a capture particle linked to CD20 antigen.

90. The kit of claim 87, wherein the kit is for monitoring treatment of a patient with a treatment ligand that binds specifically to CD52 antigen and comprises
a) a first distinguishable capture particle linked to a CD52 antigen; and
b) a first soluble ligand that binds specifically to the expressed target at the binding site of the treatment ligand conjugated to a first distinguishable fluorescent label.

91. The kit of claim 87, wherein the kit is for monitoring heparin therapy of a patient and comprises:
a) a distinguishable capture particle linked to a heparin:platelet factor 4 complex; and one or more of the following:
b) a first soluble ligand that binds specifically to a platelet activation antigen;
c) a second soluble ligand that binds specifically to platelets; and
d) a third soluble ligand that binds specifically to human immunoglobulins.

92. The method of claim 63, wherein said capture medium is a physiologically compatible bead.

93. A method for monitoring treatment of a patient in need thereof with a treatment ligand that binds specifically to the cell surface expressed antigen, comprising:
a) obtaining a container containing a sample comprising bodily fluid containing the antigen obtained from the patient;
b) performing one of the following:
i) incubating assay components in the container with a first soluble ligand that binds specifically to a cell surface antigen and is conjugated to a first distinguishable fluorescent label under conditions and for a time to allow formation of complexes of target and the first ligand; or
ii) adding to the container a second soluble ligand that binds specifically to a defined cell lineage specific antigen and is conjugated to a second fluorescent label under conditions and for a time to allow formation of complexes between assay components; or
iii) the following combination of steps:
adding to the container a capture particle linked to the target-antigen; and
incubating assay components in the container with a third soluble ligand distinct from the first soluble ligand and that binds specifically to a separate, distinct epitope on the cell surface antigen and is conjugated to a third distinguishable fluorescent label under conditions and for a time to allow formation of complexes of cell surface antigen and the third ligand; and
c) detecting the presence of fluorescence from fluorescent labels in complexes formed in the container to monitor the treatment of the patient.

94. A method for monitoring treatment of a patient in need thereof with a treatment ligand that binds specifically to intracellular expressed target antigen, comprising:
a) obtaining a container containing a sample comprising bodily fluid containing antigen obtained from the patient;
b) performing one of the following:
i) adding to the container a first soluble ligand that binds specifically to a defined cell lineage specific antigen and is conjugated to a first fluorescent label under conditions and for a time to allow formation of complexes between assay components; or
ii) the following combination of steps:
adding to the container a capture particle linked to antigen;
permeabilizing cells in the container; and
iii) incubating assay components in the container with a second soluble ligand that binds specifically to the intracellular antigen of interest and is conjugated to a second distinguishable fluorescent label under conditions and for a time to allow formation of complexes of and the second ligand; or
iv) incubating assay components in the container with a third soluble ligand distinct from the first soluble ligand that also binds specifically to a separate, distinct epitope on the intracellular antigen of interest and is conjugated to a third distinguishable fluorescent label under conditions and for a time to allow formation of complexes of the intracellular antigen and the third ligand; and
c) detecting the presence of fluorescence from fluorescent labels in complexes formed in the container to monitor the treatment of the patient.

95. The method as in claim 93 or 94, wherein the antigen is CD20 or CD52.
